SEQUENCE LISTING (49 sequences)

SEQ ID NO: 1 - DNA, 1984 bp, Artificial Sequence
  rpsL/tetA
tcgagcacgtctctatggaaatatgacggtgttcacaaagttccttaaattttacttttggttacatattttttctttttgaaaccaaatctttatctttgtagcactttcacggtagcgaaacgttagtttgaatggaaagatgcctgcagacacataaagacaccaaactctcatcaatagttccgtaaatttttattgacagaacttattgacggcagtggcaggtgtcataaaaaaaaccatgagggtaataaataatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaatggttctccaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctcga

SEQ ID NO: 2 - DNA, 20 bp, Artificial Sequence
  Forward primer
gcaggagagaggttgtgagg

SEQ ID NO: 3 - DNA, 20 bp, Artificial Sequence
  Reverse primer
gtgacccattcgagtgtcct

SEQ ID NO: 4 - DNA, 20 bp, Artificial Sequence
  Forward primer
actggtccctggtgccttat

SEQ ID NO: 5 - DNA, 20 bp, Artificial Sequence
  Reverse primer
ccttgagcaagacccagtgt

SEQ ID NO: 6 - DNA, 20 bp, Artificial Sequence
  Forward primer
gaactggggcatctctcgga

SEQ ID NO: 7 - DNA, 20 bp, Artificial Sequence
  Reverse primer
tgactggactcgcagggttt

SEQ ID NO: 8 - DNA, 22 bp, Artificial Sequence
  Forward primer
gtcccttttgctggctttggtc

SEQ ID NO: 9 - DNA, 21 bp, Artificial Sequence
  Reverse primer
ggtggccccataacacaccta

SEQ ID NO: 10 - DNA, 20 bp, Artificial Sequence
  Forward primer
tccagaagtggaagcgttta

SEQ ID NO: 11 - DNA, 22 bp, Artificial Sequence
  Reverse primer
aaaccccctggaaatcatagta

SEQ ID NO: 12 - DNA, 20 bp, Artificial Sequence
  Forward primer
ctctctctggttcccagcac

SEQ ID NO: 13 - DNA, 20 bp, Artificial Sequence
  Reverse primer
ggcaggctgactttcactct

SEQ ID NO: 14 - DNA, 20 bp, Artificial Sequence
  Forward primer
ctgagggccgatggtactaa

SEQ ID NO: 15 - DNA, 20 bp, Artificial Sequence
  Reverse primer
acactctggggccatgtaag

SEQ ID NO: 16 - DNA, 20 bp, Artificial Sequence
  Forward primer
ctaggccctggtaaccaaca

SEQ ID NO: 17 - DNA, 20 bp, Artificial Sequence
  Reverse primer
agttctgaatggggctgaga

SEQ ID NO: 18 - DNA, 20 bp, Artificial Sequence
  Forward primer
aggcatctcggcaaaaatta

SEQ ID NO: 19 - DNA, 20 bp, Artificial Sequence
  Reverse primer
ggcatggaggaaatgacaaa

SEQ ID NO: 20 - DNA, 20 bp, Artificial Sequence
  Forward primer
gggcatggacatagcagatt

SEQ ID NO: 21 - DNA, 20 bp, Artificial Sequence
  Reverse primer
gcgcaatgaactggtacaaa

SEQ ID NO: 22 - DNA, 20 bp, Artificial Sequence
  Forward primer
gcccactccacaattcctaa

SEQ ID NO: 23 - DNA, 20 bp, Artificial Sequence
  Reverse primer
ctgtgactttccccacaggt

SEQ ID NO: 24 - DNA, 20 bp, Artificial Sequence
  Forward primer
ggtgttgcatctgtggtgag

SEQ ID NO: 25 - DNA, 20 bp, Artificial Sequence
  Reverse primer
ggcttctctggaaatgcaag

SEQ ID NO: 26 - DNA, 20 bp, Artificial Sequence
  Forward primer
agcgaaaggagtcattcaaa

SEQ ID NO: 27 - DNA, 20 bp, Artificial Sequence
  Reverse primer
ggttggtttccaggttgtgt

SEQ ID NO: 28 - DNA, 20 bp, Artificial Sequence
  Forward primer
ttttgctccttcctgtgtcc

SEQ ID NO: 29 - DNA, 20 bp, Artificial Sequence
  Reverse primer
atccagcaccacagtcacaa

SEQ ID NO: 30 - DNA, 20 bp, Artificial Sequence
  Forward primer
aacaaaagcaggcgttcact

SEQ ID NO: 31 - DNA, 20 bp, Artificial Sequence
  Reverse primer
cacccatccactgcctattt

SEQ ID NO: 32 - DNA, 20 bp, Artificial Sequence
  Forward primer
ctcagtaagggagcgcatct

SEQ ID NO: 33 - DNA, 19 bp, Artificial Sequence
  Reverse primer
gggctgagaaaagggaagt

SEQ ID NO: 34 - DNA, 20 bp, Artificial Sequence
  Forward primer
atggggcacaaaggtatgtt

SEQ ID NO: 35 - DNA, 20 bp, Artificial Sequence
  Reverse primer
ccagtgtggtctcgatttcc

SEQ ID NO: 36 - DNA, 20 bp, Artificial Sequence
  Forward primer
agggtcccagataggttgct

SEQ ID NO: 37 - DNA, 20 bp, Artificial Sequence
  Reverse primer
cctgaaagatcgggctgtaa

SEQ ID NO: 38 - DNA, 20 bp, Artificial Sequence
  Forward primer
gctccctaccatccattcaa

SEQ ID NO: 39 - DNA, 20 bp, Artificial Sequence
  Reverse primer
gttcaaacaaaaggcccaga

SEQ ID NO: 40 - DNA, 20 bp, Artificial Sequence
  Forward primer
ttactttgcaggggaaccac

SEQ ID NO: 41 - DNA, 20 bp, Artificial Sequence
  Reverse primer
tgagtgttcctgaccctcct

SEQ ID NO: 42 - DNA, 21 bp, Artificial Sequence
  Forward primer
gcaaatgctgtttatggatca

SEQ ID NO: 43 - DNA, 20 bp, Artificial Sequence
  Reverse primer
gcaaatggcagcatctttct

SEQ ID NO: 44 - DNA, 20 bp, Artificial Sequence
  Forward primer
ctctcacccagggaaaacag

SEQ ID NO: 45 - DNA, 22 bp, Artificial Sequence
  Reverse primer
gataaccagacatgttgggtca

SEQ ID NO: 46 - DNA, 20 bp, Artificial Sequence
  Forward primer
ccttgctaggttggggaagt

SEQ ID NO: 47 - DNA, 20 bp, Artificial Sequence
  Reverse primer
ccagcaacagaacaaagctg

SEQ ID NO: 48 - DNA, 20 bp, Artificial Sequence
  Forward primer
ggtgagaggcctttggagat

SEQ ID NO: 49 - DNA, 20 bp, Artificial Sequence
  Reverse primer
catcacaccatgttcccatt

(19) United States
(12) Patent Application Publication
(10) Pub. No.: US 2024/0130339 A1
(43) Pub. Date: Apr. 25, 2024

(54) RECOMBINANT GENOME, AND NON-HUMAN MAMMALIAN CELL AND PRODUCTION METHOD THEREFOR AND USE THEREOF

(71) Applicant: SHANGHAI ACEMAB CORPORATION LTD, Shanghai (CN)

(72) Inventors: Seehong WONG, Shanghai (CN); Jianliang LI, Shanghai (CN)

(21) Appl. No.: 18/270,023

(22) PCT Filed: Jan. 13, 2022
(86) PCT No.: PCT/CN22/71889

§ 371 Date: Jun. 27, 2023

(30) Foreign Application Priority Data
Jan. 14, 2021 (CN) 202110051015.0

(51) Int. Cl. A01K 67/0278; C07K 16/46; C12N 9/22; C12N 15/85; C12N 15/90

(52) U.S. Cl. CPC A01K 67/0278; C07K 16/464; C12N 9/22; C12N 15/8509; C12N 15/907; A01K 2207/15; A01K 2217/072; A01K 2227/105; A01K 2267/01; C07K 2317/14; C07K 2317/24; C12N 2015/8518; C12N 2800/107; C12N 2800/90

(57) ABSTRACT

Disclosed are a non-human mammalian cell, a recombinant genome thereof, and a method for producing same. The variable region genes of the endogenous immunoglobulin in the genome are partially or entirely replaced with variable region genes of human immunoglobulin, a part or all of the pseudogenes and/or open reading frames of the variable region genes of human immunoglobulin, including coding and non-coding sequences for human heavy chain functional V_{H}, D_{H}, J_{H}, or coding and non-coding sequences for human light chain functional V_{L}, J_{L}, are knocked out. A non-human mammalian cell of the present invention that contains human immunoglobulin domains can be used to produce a transgenic animal that is capable of producing antibodies with fully human variable domain(s), whereby fully human antibodies featuring a higher affinity can be screened efficiently at a lower cost within a shorter period.

### TECHNICAL FIELD

The present invention relates to the field of genetic engineering, in particular to genetically engineered non-human mammalian cell and the genome thereof for medical and disease research, a method for obtaining a non-human mammal based on the non-human mammalian cell and the genome thereof, as well as a cell, antibody, antibody fragment and derivative drug or pharmaceutical composition comprising the antibody fragment derived from such animal.

### BACKGROUND

Bruggemann et al. 1989a first reported that introduction of unrearranged human immunoglobulin gene segments in an animal resulted in detection of antibodies derived from human immunoglobulin genes in the serum of the animal, and such an attempt opened the chapter to utilize genetically engineered animal to directly generate therapeutic antibody with fully human variable region(s) in vivo. Many companies produce transgenic animals bearing human immunoglobulin genes based on similar principles, and these preparation methods and examples are described in International Applications WO90/10077, WO90/04036, WO2012/018610, WO2010/039900, WO2011/004192, WO2002/066630, WO1994/002602, WO1996/030498, WO1998/024893, WO1994/004667, WO1990/006359, WO1992/003917, US Applications US7041871, U.S. Pat. No. 6,673,986, US6091001, U.S. Pat. No. 5,877,397 and Nat Biotechnol. 32 (4): 356-63, Proc Natl Acad Sci USA. 111 (14): 5147-52 and Proc Natl Acad Sci USA. 111 (14): 5153-8.

These methods involve inactivation of endogenous antibody gene cluster functions in the animal, and recombination and expression of human immunoglobulin genes. Genetic engineering to achieve these is typically performed in these non-human mammalian embryonic stem cells, for example, knocking out a part or all of the heavy and light chain loci in mouse embryonic stem cells whilst introducing human heavy and light chain loci to compensate for the loss of functions of such genes results in the mouse producing antibodies derived from human heavy and light chain gene fragments. However, in the prior art, such animal models are time and cost consuming and often have some limitations or drawbacks:
1) The size of endogenous immunoglobulin loci in animals that need to be knocked out or inactivated is generally Megabase scale, which often results in low efficiency and success rate during genetic engineering. Incomplete knockout or inactivation of endogenous immunoglobulin loci will result in obtained animals expressing human antibodies as well as endogenous murine antibodies, increasing the challenges of antibody screening;
2) The size of the human immunoglobulin loci that need to be inserted is also Megabase scale, while the vector size limits the size of the human DNA fragments that is introduced at one time, often requiring more steps and a longer period of time to introduce all human immunoglobulin gene fragments in batches. Animals obtained without total introduction have only a small V region repertoire or fewer constant region classes, and thus can only possess a small diversity of human-derived antibodies or poor B cell development;
3) The efficiency of successful insertion of large DNA fragments of human-derived immunoglobulin loci, particularly in situ insertions, is extremely low, and factors of low efficiency increase the cost of time and risk of failure to obtain such transgenic animals when these insertion steps need to be performed multiple times. When the random insertion method is used to introduce large DNA fragments of human immunoglobulin loci into such transgenic animals, some mouse models show variable arrest of B cell development due to the uncertainty resulted from the deletion of long-range regulatory regions and the random insertion sites, and in particular, the process of T1-type B cell development to T2-type B cell is delayed. The level of immune response of these animals to the antigen is difficult to reach that of non-transgenic animals, and the affinity of antibodies generated is difficult to reach that of antibodies generated in non-transgenic animals;
4) Due to the constraints of the size, quantity and the like of human immunoglobulin gene fragments introduced, the number of transgenic lines that can be optimized for expression analysis is limited, there is low efficient V (D) J recombination and partial gene complementation, such that the obtained transgenic mice typically produce a limited number of antibodies, resulting in inefficient antibody production.

Based on the analysis on the above-mentioned limitations, it can be seen that, there is a need for low cost and fast methods of obtaining a large repertoire of antibodies, and high efficiency of rearrangement and expression of human variable region gene fragments, and in particular, for transgenic animals that have good antigen response capability and can efficiently express high affinity humanized immunoglobulins.

### SUMMARY OF THE INVENTION

To address the aforementioned deficiencies of the prior art, the present invention provides a stably inheritable non-human mammalian cell for fully human therapeutic antibody screening, the genome thereof may comprise 41 human immunoglobulin heavy chain variable region functional V genes, or 20 human immunoglobulin kappa light chain variable region functional V genes, or 31 human immunoglobulin lambda light chain variable region functional V genes. Transgenic animals prepared based on such non-human mammalian cells are highly efficient and rapid in obtaining high diversity and high affinity of antibodies with fully human variable regions.

The inventors analyzed the human genome database, wherein human heavy chain variable region DNA fragments are derived from positions 105863198 to 106879844 on human chromosome 14, human Kappa light chain variable region DNA fragments are derived from positions 88860568 to 90235398 on human chromosome 2, and human Lambda light chain variable region DNA fragments are derived from positions 22023114 to 22922913 on human chromosome 22, all coordinates refer to the GRCh38.p13 version of the human genome database from ENSEMBL.

The inventors have found that in human immunoglobulin loci, the V-region gene, D-region gene, J-region gene or constant region gene may have pseudogenes or open reading frames that do not or only inefficiently contribute into the rearranged antibody transcriptome. The definitions and characteristics of these three classes of genes refer to the following IMGT database illustrative link:
http://www.imgt.org/IMGTScientificChart/SequenceDescripton/IMGTfunctionality.html.

By selecting Homo sapiens species and gene classes (variable for V region gene, diversity for D region gene, joining for J region gene, constant for constant region gene) along with the corresponding immunoglobulin locus name (IGH for heavy chain locus, IGK for Kappa light chain locus, IGL for Lambda light chain locus) in database link http: //www.imgt.org/genedb/, we can find the list of gene names for functional genes, pseudogenes, and open reading frames (ORFs) in human immunoglobulin loci. Notably, all reported functional genes, pseudogenes, or open reading frame genes are included in the IMGT database, while in real fact, some of the genes in the database are likely to be present in only a small number of individuals.

The gene fragments of the human immunoglobulin heavy chain locus, Kappa light chain, and Lambda light chain locus can be cloned into BAC vectors or YAC vectors that can replicate in E. coli or yeast by a method of BAC or YAC library construction. Without gene editing, pseudo-V-genes, open reading frame V-genes, and functional V-gene segments are hybrid arranged (as shown in FIG. 1). These genes may each include three regions, a gene regulatory region at the 5 ′end, a gene coding sequence region (including introns and exons), and an antibody gene Recombination Signal Sequence (RSS) at the 3′ end. The locations for some human functional V-gene, pseudo-V-gene, and open reading frame gene fragments (including the 5 ′end regulatory region, gene coding sequence region, and 3′ end antibody gene Recombination Signal Sequence (RSS) of the V-region genes) in genome are as shown in Table 1, and the coordinates refer to the GRCh38.p13 version of the human genome database from ENSEMBL.

TABLE 1
List of human heavy chain V region genes, human Kappa light chain proximal V region
genes and human Lambda light chain V region genes and categories thereof position

| | | | Start position | End position | Fragment |
|---|---|---|---|---|---|
| Sequence | Name of human | Category of | of human | of human | size (base |
| number | heavy chain V gene | V gene | chromosome 14 | chromosome 14 | pairs) |
| H1 | IGHV6-1 | functional V gene | 105944896 | 105939715 | 5182 |
| H2 | IGHV(II)-1-1 | pseudo-V-gene | 105986542 | 105944897 | 41646 |
| H3 | IGHV1-2 | functional V gene | 106001494 | 105986543 | 14952 |
| H4 | IGHV(III)-2-1 | pseudo-V-gene | 106005055 | 106001495 | 3561 |
| H5 | IGHV1-3 | functional V gene | 106011882 | 106005056 | 6827 |
| H6 | IGHV4-4 | functional V gene | 106025105 | 106011883 | 13223 |
| H7 | IGHV7-4-1 | functional V gene | 106037862 | 106025106 | 12757 |
| H8 | IGHV2-5 | functional V gene | 106039632 | 106037863 | 1770 |
| H9 | IGHV(III)-5-1, | pseudo-V-gene | 106062109 | 106039633 | 22477 |
| | IGHV(III)-5-2, | | | | |
| | IGHV3-6 | | | | |
| H10 | IGHV3-7, | functional V gene | 106088082 | 106062110 | 25973 |
| | IGHV1-8, | | | | |
| | IGHV3-9 | | | | |
| H11 | IGHV2-10 | pseudo-V-gene | 106116595 | 106088083 | 28513 |
| H12 | IGHV3-11 | functional V gene | 106120286 | 106116596 | 3691 |
| H13 | IGHV(III)-11-1, | pseudo-V-gene | 106129500 | 106120287 | 9214 |
| | IGHV1-12 | | | | |
| H14 | IGHV3-13 | functional V gene | 106142246 | 106129501 | 12746 |
| H15 | IGHV(III)-13-1, | pseudo-V-gene | 106153582 | 106142247 | 11336 |
| | IGHV1-14 | | | | |
| H16 | IGHV3-15 | functional V gene | 106163495 | 106153583 | 9913 |
| H17 | IGHV(II)-15-1, | pseudo-V-gene or | 106184859 | 106163496 | 21364 |
| | IGHV(II)-16-1, | open reading frame | | | |
| | IGHV1-17, | | | | |
| | IGHV1-16(ORF) | | | | |
| H18 | IGHV1-18 | functional V gene | 106196658 | 106184860 | 11799 |
| H19 | IGHV3-19 | pseudo-V-gene | 106210896 | 106196659 | 14238 |
| H20 | IGHV3-20 | functional V gene | 106212785 | 106210897 | 1889 |
| H21 | IGHV(II)-20-1 | pseudo-V-gene | 106235022 | 106212786 | 22237 |
| H22 | IGHV3-21 | functional V gene | 106257722 | 106235023 | 22700 |
| H23 | IGHV3-22, | pseudo-V-gene | 106268566 | 106257723 | 10844 |
| | IGHV(II)-22-1, | | | | |
| | IGHV(III)-22-2 | | | | |
| H24 | IGHV3-23 | functional V gene | 106276506 | 106268567 | 7940 |
| H25 | IGHV1-24 | functional V gene | 106288923 | 106276507 | 12417 |
| H26 | IGHV3-25, | pseudo-V-gene | 106301355 | 106288924 | 12432 |
| | IGHV(III)-25-1 | | | | |
| H27 | IGHV2-26 | functional V gene | 106308995 | 106301356 | 7640 |
| H28 | IGHV(III)-26-1, | pseudo-V-gene | 106324214 | 106308996 | 15219 |
| | IGHV(II)-26-2, | | | | |
| | IGHV7-27 | | | | |
| H29 | IGHV4-28 | functional V gene | 106331105 | 106324215 | 6891 |
| H30 | IGHV3-29 | pseudo-V-gene | 106335040 | 106331106 | 3935 |
| H31 | IGHV3-30 | functional V gene | 106344384 | 106335041 | 9344 |
| H32 | IGHV3-30-2 | pseudo-V-gene | 106349243 | 106344385 | 4859 |
| H33 | IGHV4-31 | functional V gene | 106356144 | 106349244 | 6901 |
| H34 | IGHV3-32 | pseudo-V-gene | 106359751 | 106356145 | 3607 |
| H35 | GOLGA4P1 | other unrelated gene | 106360243 | 106359752 | 492 |
| H36 | IGHV3-33 | functional V gene | 106369097 | 106360244 | 8854 |
| H37 | IGHV3-33-2 | pseudo-V-gene | 106373621 | 106369098 | 4524 |
| H38 | IGHV4-34 | functional V gene | 106377231 | 106373622 | 3610 |
| H39 | IGHV7-34-1, | pseudo-V-gene or | 106421669 | 106377232 | 44438 |
| | IGHV3-35(ORF), | open reading frame | | | |
| | IGHV3-36, | | | | |
| | IGHV3-37, | | | | |
| | IGHV3-38(ORF), | | | | |
| | IGHV(III)-38-1 | | | | |
| H40 | IGHV4-39 | functional V gene | 106425408 | 106421670 | 3739 |
| H41 | IGHV7-40, | pseudo-V-gene | 106470223 | 106425409 | 44815 |
| | IGHV(II)-40-1, | | | | |
| | IGHV3-41, | | | | |
| | IGHV3-42 | | | | |
| H42 | IGHV3-43 | functional V gene | 106472846 | 106470224 | 2623 |
| H43 | IGHV(II)-44-1, | pseudo-V-gene | 106506956 | 106472847 | 34110 |
| | IGHV(III)-44, | | | | |
| | IGHV(IV)-44-1, | | | | |
| | IGHV(II)-44-2 | | | | |
| H44 | IGHV1-45 | functional V gene | 106511075 | 106506957 | 4119 |
| H45 | IGHV1-46 | functional V gene | 106515891 | 106511076 | 4816 |
| H46 | IGHV(II)-46-1, | pseudo-V-gene | 106537770 | 106515892 | 21879 |
| | IGHV3-47, | | | | |
| | IGHV(III)-47-1 | | | | |
| H47 | IGHV3-48 | functional V gene | 106556896 | 106537771 | 19126 |
| H48 | IGHV3-49 | functional V gene | 106564377 | 106556897 | 7481 |
| H49 | IGHV(II)-49-1, | pseudo-V-gene | 106578702 | 106564378 | 14325 |
| | IGHV3-50 | | | | |
| H50 | IGHV5-51 | functional V gene | 106583409 | 106578703 | 4707 |
| H51 | IGHV8-51-1, | pseudo-V-gene | 106592636 | 106583410 | 9227 |
| | IGHV(II)-51-2, | | | | |
| | IGHV3-52 | | | | |
| H52 | IGHV3-53 | functional V gene | 106599595 | 106592637 | 6959 |
| H53 | IGHV(II)-53-1, | pseudo-V-gene | 106622317 | 106599596 | 22722 |
| | IGHV3-54, | | | | |
| | IGHV4-55, | | | | |
| | IGHV7-56, | | | | |
| | IGHV3-57 | | | | |
| H54 | IGHV1-58 | functional V gene | 106627209 | 106622318 | 4892 |
| H55 | IGHV4-59 | functional V gene | 106639079 | 106627210 | 11870 |
| H56 | IGHV4-61 | functional V gene | 106643021 | 106639080 | 3942 |
| H57 | IGHV3-62, | pseudo-V-gene | 106657683 | 106643022 | 14662 |
| | IGHV(II)-62-1, | | | | |
| | IGHV3-63 | | | | |
| H58 | IGHV3-64 | functional V gene | 106666004 | 106657684 | 8321 |
| H59 | IGHV3-65, | pseudo-V-gene | 106674975 | 106666005 | 8971 |
| | IGHV(II)-65-1 | | | | |
| H60 | IGHV3-66 | functional V gene | 106680592 | 106674976 | 5617 |
| H61 | IGHV(III)-67-1, | pseudo-V-gene | 106762052 | 106680593 | 81460 |
| | IGHV(III)-67-2, | | | | |
| | IGHV(III)-67-3, | | | | |
| | IGHV(III)-67-4, | | | | |
| | IGHV1-68 | | | | |
| H62 | IGHV1-69 | functional V gene | 106770537 | 106762053 | 8485 |
| H63 | IGHV2-70 | functional V gene | 106775077 | 106770538 | 4540 |
| H64 | IGHV3-71 | pseudo-V-gene | 106790652 | 106775078 | 15575 |
| H65 | IGHV3-72 | functional V gene | 106802652 | 106790653 | 12000 |
| H66 | IGHV3-73 | functional V gene | 106810400 | 106802653 | 7748 |
| H67 | IGHV3-74 | functional V gene | 106822782 | 106810401 | 12382 |
| H68 | IGHV(II)-74-1, | pseudo-V-gene or | 106879844 | 106822783 | 57062 |
| | IGHV3-75, | open reading frame | | | |
| | IGHV3-76, | | | | |
| | IGHV5-78, | | | | |
| | IGHV(II)-78-1, | | | | |
| | IGHV3-79, | | | | |
| | IGHV4-80, | | | | |
| | IGHV7-81(ORF), | | | | |
| | IGHV(III)-82 | | | | |

| | Name of human | | Start position | End position | Fragment |
|---|---|---|---|---|---|
| Sequence | kappa light chain | Category of | of human | of human | size (base |
| number | proximal V Gene | V gene | chromosome 2 | chromosome 2 | pairs) |
| K1 | IGKV4-1 | functional V gene | 88861968 | 88886183 | 24216 |
| K2 | IGKV5-2 | functional V gene | 88886184 | 88897814 | 11631 |
| K3 | IGKV7-3, | pseudo-V-gene | 88947270 | 88897815 | 49456 |
| | IGKV2-4 | | | | |
| K4 | IGKV1-5 | functional V gene | 88966231 | 88947271 | 18961 |
| K5 | IGKV1-6 | functional V gene | 88978388 | 88966232 | 12157 |
| K6 | IGKV3-7 | open reading frame | 88992378 | 88978389 | 13990 |
| K7 | IGKV1-8 | functional V gene | 89009981 | 88992379 | 17603 |
| K8 | IGKV1-9 | functional V gene | 89019913 | 89009982 | 9932 |
| K9 | IGKV2-10 | pseudo-V-gene | 89027140 | 89019914 | 7227 |
| K10 | IGKV3-11 | functional V gene | 89040193 | 89027141 | 13053 |
| K11 | IGKV1-12 | functional V gene | 89045910 | 89040194 | 5717 |
| K12 | IGKV1-13, | pseudo-V-gene | 89085146 | 89045911 | 39236 |
| | IGKV2-14 | | | | |
| K13 | IGKV3-15 | functional V gene | 89099828 | 89085147 | 14682 |
| K14 | IGKV1-16 | functional V gene | 89117311 | 89099829 | 17483 |
| K15 | IGKV1-17 | functional V gene | 89128657 | 89117312 | 11346 |
| K16 | IGKV2-18, | pseudo-V-gene | 89142543 | 89128658 | 13886 |
| | IGKV2-19 | | | | |
| K17 | IGKV3-20 | functional V gene | 89159022 | 89142544 | 16479 |
| K18 | IGKV6-21 | functional V gene | 89170744 | 89159023 | 11722 |
| K19 | IGKV1-22, | pseudo-V-gene | 89176297 | 89170745 | 5553 |
| | IGKV2-23 | | | | |
| K20 | IGKV2-24 | functional V gene | 89192412 | 89176298 | 16115 |
| K21 | IGKV3-25, | pseudo-V-gene | 89213392 | 89192413 | 20980 |
| | IGKV2-26 | | | | |
| K22 | IGKV1-27 | functional V gene | 89221667 | 89213393 | 8275 |
| K23 | IGKV2-28 | functional V gene | 89234120 | 89221668 | 12453 |
| K24 | IGKV2-29 | pseudo-V-gene | 89244750 | 89234121 | 10630 |
| K25 | IGKV2-30 | functional V gene | 89252117 | 89244751 | 7367 |
| K26 | IGKV3-31, | pseudo-V-gene | 89267970 | 89252118 | 15853 |
| | IGKV1-32 | | | | |
| K27 | IGKV1-33 | functional V gene | 89275176 | 89267971 | 7206 |
| K28 | IGKV3-34, | pseudo-V-gene or | 89319594 | 89275177 | 44418 |
| | IGKV1-35, | open reading frame | | | |
| | IGKV2-36, | | | | |
| | IGKV1-37(ORF), | | | | |
| | IGKV2-38 | | | | |
| K29 | IGKV1-39 | functional V gene | 89330085 | 89319595 | 10491 |
| K30 | IGKV2-40 | functional V gene | 89333431 | 89330086 | 3346 |

| | Name of human | | Start position | End position | Fragment |
|---|---|---|---|---|---|
| Sequence | lambda light | Category of | of human | of human | size (base |
| number | chain V Gene | V gene | chromosome 22 | chromosome 22 | pairs) |
| L1 | IGLV3-1 | functional V gene | 22873533 | 22881431 | 7898 |
| L2 | IGLV3-2 | pseudo-V-gene | 22872075 | 22873532 | 1457 |
| L3 | IGLV4-3 | functional V gene | 22857100 | 22872074 | 14974 |
| L4 | IGLV3-4, | pseudo-V-gene | 22823329 | 22857099 | 33770 |
| | IGLV2-5, | | | | |
| | IGLV3-6, | | | | |
| | IGLV3-7 | | | | |
| L5 | IGLV2-8 | functional V gene | 22819792 | 22823328 | 3536 |
| L6 | IGLV3-9 | functional V gene | 22812321 | 22819791 | 7470 |
| L7 | IGLV3-10 | functional V gene | 22793043 | 22812320 | 19277 |
| L8 | IGLV2-11 | functional V gene | 22772622 | 22793042 | 20420 |
| L9 | IGLV3-12 | functional V gene | 22762614 | 22772621 | 10007 |
| L10 | IGLV3-13 | pseudo-V-gene | 22759251 | 22762613 | 3362 |
| L11 | IGLV2-14 | functional V gene | 22755877 | 22759250 | 3373 |
| L12 | IGLV3-15 | pseudo-V-gene | 22747961 | 22755876 | 7915 |
| L13 | IGLV3-16 | functional V gene | 22739231 | 22747960 | 8729 |
| L14 | IGLV3-17 | pseudo-V-gene | 22735129 | 22739230 | 4101 |
| L15 | IGLV2-18 | functional V gene | 22721195 | 22735128 | 13933 |
| L16 | IGLV3-19 | functional V gene | 22715483 | 22721194 | 5711 |
| L17 | IGLV(I)-20 | pseudo-V-gene | 22713239 | 22715482 | 2243 |
| L18 | IGLV3-21 | functional V gene | 22704858 | 22713238 | 8380 |
| L19 | IGLV3-22 | pseudo-V-gene | 22698461 | 22704857 | 6396 |
| L20 | IGLV2-23 | functional V gene | 22695110 | 22698460 | 3350 |
| L21 | IGLV3-24, | pseudo-V-gene | 22687321 | 22695109 | 7788 |
| | IGLV(II)-24-1 | | | | |
| L22 | IGLV3-25 | functional V gene | 22685389 | 22687320 | 1931 |
| L23 | IGLV(VI)-25-1, | pseudo-V-gene | 22668848 | 22685388 | 16540 |
| | IGLV3-26 | | | | |
| L24 | IGLV3-27 | functional V gene | 22664971 | 22668847 | 3876 |
| L25 | IGLV3-28, | pseudo-V-gene or | 22432505 | 22664970 | 232465 |
| | IGLV3-29, | open reading frame | | | |
| | IGLV3-30, | | | | |
| | IGLV3-31, | | | | |
| | IGLV3-32(ORF), | | | | |
| | IGLV2-33(ORF), | | | | |
| | IGLV2-34, | | | | |
| | IGLV7-35 | | | | |
| L26 | IGLV1-36 | functional V gene | 22428075 | 22432504 | 4429 |
| L27 | IGLV5-37 | functional V gene | 22426318 | 22428074 | 1756 |
| L28 | IGLV(I)-38 | pseudo-V-gene | 22410322 | 22426317 | 15995 |
| L29 | IGLV1-40 | functional V gene | 22404761 | 22410321 | 5560 |
| L30 | IGLV1-41(ORF), | pseudo-V-gene or | 22395529 | 22404760 | 9231 |
| | IGLV(I)-42 | open reading frame | | | |
| L31 | IGLV7-43 | functional V gene | 22381387 | 22395528 | 14141 |
| L32 | IGLV1-44 | functional V gene | 22376545 | 22381386 | 4841 |
| L33 | IGLV5-45 | functional V gene | 22370127 | 22376544 | 6417 |
| L34 | IGLV7-46 | functional V gene | 22358302 | 22370126 | 11824 |
| L35 | IGLV1-47 | functional V gene | 22353473 | 22358301 | 4828 |
| L36 | GLV5-48 | open reading frame | 22343774 | 22353472 | 9698 |
| L37 | IGLV9-49 | functional V gene | 22327856 | 22343773 | 15917 |
| L38 | GLV1-50 | open reading frame | 22323009 | 22327855 | 4846 |
| L39 | IGLV1-51 | functional V gene | 22319266 | 22323008 | 3742 |
| L40 | IGLV5-52 | functional V gene | 22220173 | 22319265 | 99092 |
| L41 | IGLV(IV)-53 | pseudo-V-gene | 22215311 | 22220172 | 4861 |
| L42 | IGLV10-54 | functional V gene | 22202201 | 22215310 | 13109 |
| L43 | IGLV11-55(ORF), | pseudo-V-gene or | 22196316 | 22202200 | 5884 |
| | IGLV(I)-56 | open reading frame | | | |
| L44 | IGLV6-57 | functional V gene | 22182891 | 22196315 | 13424 |
| L45 | IGLV(V)-58, | pseudo-V-gene | 22162721 | 22182890 | 20169 |
| | IGLV(IV)-59, | | | | |
| | IGLV(III)-59-1 | | | | |
| L46 | IGLV4-60 | functional V gene | 22099252 | 22162720 | 63468 |
| L47 | IGLV8-61 | functional V gene | 22087064 | 22099251 | 12187 |
| L48 | IGLV1-62, | pseudo-V-gene | 22031512 | 22087063 | 55551 |
| | IGLV(I)-63, | | | | |
| | IGLV(IV)-64, | | | | |
| | IGLV(IV)-65, | | | | |
| | IGLV(V)-66, | | | | |
| | IGLV(IV)-66-1, | | | | |
| | IGLV10-67, | | | | |
| | IGLV(I)-68 | | | | |
| L49 | IGLV4-69 | functional V gene | 22026593 | 22031511 | 4918 |
| L50 | IGLVI-70 | pseudo-V-gene | 22023114 | 22026592 | 3478 |

Note:
1) the V gene was a larger start position number than the end position number is in the reverse complementary direction, and the V gene with a smaller start position number than the end position number is in the forward direction;
2) All the coordinates refer to the GRCh38.p13 version of the human genome database from ENSEMBL.

Note: 1) the V gene with a larger start position number than the end position number is in the reverse complementary direction, and the V gene with a smaller start position number than the end position number is in the forward direction; 2) All the coordinates refer to the GRCh38.p13 version of the human genome database from ENSEMBL.

Further, the inventors found that, analysis of the Gene Frequency and sequencing of the antibody cDNA transcriptome of these three classes of variable region gene fragments after rearrangement, in http://www.imgt.org/genefrequency/query database, showed that functional gene fragments can contribute to the antibody transcriptome with a high frequency through gene rearrangements, whereas pseudogenes and open reading frames rarely or never contribute to the antibody transcriptome.

While pseudogenes and open reading frames rarely or never contribute to the antibody transcriptome, these two classes of genes can still produce ineffective V/D/J or V/J rearrangement products through gene rearrangements. While B cells with unproductive VDJ rearrangement will eventually be eliminated by apoptosis, the existence of a large number of pseudogenes and open reading frames could one of the reasons behind the non-functional V/D/J or V/J rearrangement. Therefore, the inventors speculate that the indiscriminate introduction of pseudogenes and open reading frames (ORF) into the animal genome increases the cost of trial and error of human-derived variable region gene segments rearrangement in the animal and decreases the recombination efficiency.

Using human, mouse, or rat immunoglobulin variable region loci as an example, functional genes, pseudogenes, and open reading frames are interspersed, and both pseudogenes and open reading frames may cause interference with effective rearrangements during gene rearrangement, gene transcription, and even translation stages, resulting inreduced efficiency of productive rearrangement. Since the pseudogenes and open reading frames do not contribute to the final antibody repertoire, the inventors attempt to knockout these genes at the genomic level, in order to meet the need for efficient production of human antibodies.

In one aspect, the present invention provides a nucleic acid construct (or a non-human mammalian genetically engineered recombinant genome), the nucleic acid construct (or recombinant genome) comprises variable region gene segments of human immunoglobulin loci, and a part (i.e., one or more) or all of the pseudo-V-genes and/or the open reading frame genes in the variable region gene segments of the human immunoglobulin loci are deleted.

In the present application, “part or all” “partially or entirely” means one or more or all.

Both the coding and non-coding regions for the variable region gene segments of the human immunoglobulin loci are from human immunoglobulins.

Specifically, the present invention provides a non-human mammalian genetically engineered recombinant genome (or, a nucleic acid construct), wherein the endogenous immunoglobulin variable region genes are partially or entirely replaced with human immunoglobulin variable region genes having part or all of the pseudogene and/or open reading frames knocked out.

“Human immunoglobulin variable region gene” has the same meaning as “variable region gene (segment) of the human immunoglobulin locus”.

In the present application, optionally, other unrelated genes of the human immunoglobulin variable region genes (e.g., the genes identified by the H35 fragment in Table 1) are partially or entirely knocked out.

Optionally, the variable region of the human immunoglobulin locus is selected from the heavy chain variable region, and/or the K light chain variable region, and/or the A light chain variable region of a human immunoglobulin.

Optionally, the variable region of the human immunoglobulin locus is selected from any one or combination of V, D and J regions of human immunoglobulin heavy chain variable region.

Optionally, the variable region of the human immunoglobulin locus is selected from any one or combination of V and J regions of human immunoglobulin K light chain variable region.

Optionally, the variable region of the human immunoglobulin locus is selected from any one or combination of V and J regions of human immunoglobulin A light chain variable region.

Optionally, the pseudogenes and/or open reading frame genes are deleted by gene knockout. Optionally, the gene knockout is performed in prokaryotic or eukaryotic cells, such as bacteria, yeast, insect cells, plant cells, E. coli, CHO, Pichia, and the like.

Additionally, the definitions for pseudo-V-gene and open reading frame gene refer to the IMGT database.

In the nucleic acid construct (or genetically engineered recombinant genome) of the present invention, both the coding and non-coding regions of the variable region gene segments of a human immunoglobulin locus are derived from human immunoglobulins. Optionally, the variable region gene segments of the human immunoglobulin locus include coding and non-coding sequences for functional V, D, and J regions of human immunoglobulin heavy chain variable region. Optionally, the variable region gene segments of the human immunoglobulin locus include coding and non-coding sequences for functional V and J regions of human immunoglobulin K light chain variable region, and/or coding and non-coding sequences for functional V and J regions of the human immunoglobulin A light chain variable region.

For example, in one particular embodiment, the nucleic acid construct (or genetically engineered recombinant genome) of the present invention comprises: (1) one that 10 DNA fragments H2, H4, H9, H11, H13, H15, H19, H21, H23, H17 in the V region of human immunoglobulin heavy chain comprising pseudo-V region genes or open reading frame V region genes in Table 1 are knocked out, and H1, H3, H5, H6, H7, H8, H10, H12, H14, H16, H18, H2O, H22, H24 (which comprise functional human heavy chain V region genes IGHV6-1, IGHV1-2, IGHV1-3, IGHV4-4, IGHV7-4-1, IGHV2-5, IGHV3-7, IGHV1-8, IGHV3-9, IGHV3-11, IGHV3-13, IGHV3-15, IGHV1-18, IGHV3-20, IGHV3-21, IGHV3-23, respectively), and sequence between 105863198 and 105939714 of human chromosome 14 that containing human immunoglobulin heavy chain D gene region and J gene region are retained; or (2) one that 14 DNA fragments H26, H28, H39, H41, H43, H46, H49, H51, H53, H57, H59, H61, H64, H68 comprising pseudo-V-genes or open reading frame V-region genes as described in Table 1 are knocked out, and 25 fragments of H25, H27, H29, H31, H33, H36, H38, H40, H42, H44, H45, H47, H48, H50, H52, H54, H55, H56, H58, H60, H62, H63, H65, H66, H67 comprising functional V-region genes, and the pseudo-V-region gene fragments H30, H32, H34, H37, as well as H35 gene fragment as described in Table 1 are retained; or, (3) one that 10 regions of K3, K6, K9, K12, K16, K19, K21, K24, K26, K28 comprising the pseudo-V-gene or open reading frame as described in Table 1 are knocked out, and 20 regions of K1, K2, K4, K5, K7, K8, K10, K11, K13, K14, K15, K17, K18, K20, K22, K23, K25, K27, K29, K30 comprising the functional human Kappa light chain V region gene and the human immunoglobulin Kappa light chain J gene region between 88861967 and 88860568 of human chromosome 2 as described in Table 1 are retained; or (4) one that 12 regions of L2, L4, L10, L12, L14, L17, L19, L21, L23, L25, L28, L30 comprising pseudo-V-genes or open reading frames as described in Table 1 are knocked out, 19 regions of L1, L3, L5, L6, L7, L8, L9, L11, L13, L15, L16, L18, L20, L22, L24, L26, L27, L29, L31 comprising functional human Lambda light chain V region gene as described in Table 1 and the gene region between 22881432 and 22922913 of human chromosome number 22 comprising human immunoglobulin lambda light chain J gene region and human C gene region are retained; or, (5) one that 7 regions of L36, L38, L41, L43, L45, L48, L50 comprising the pseudo-V-gene or open reading frame as described in Table 1 are knocked out, and 12 regions of L32, L33, L34, L35, L37, L39, L40, L42, L44, L46, L47, L49 comprising the functional human Lambda light chain V region gene as described in Table 1 are retained; or one comprising any two or more of (1)-(5), preferably both (1) and (2), or both (4) and (5).

The present invention also provides a method of preparing a nucleic acid construct (or non-human mammalian genetically engineered recombinant genome), comprising:
(1) amplifying each of the variable region gene segments of the human immunoglobulin locus by PCR, optionally, further inserting each amplified fragment into a vector individually in an appropriate position, or ligating the amplified fragments partially or entirely followed by inserting into a vector in an appropriate position; or
(2) knocking out pseudo-V-genes and/or open reading frames in the heavy chain variable region DNA fragment or light chain variable region DNA fragment of a human immunoglobulin by a method of knocking out, thereby obtaining variable region gene segments of the human immunoglobulin locus; or.
(3) the method of synthesizing gene.

In the present application, the human immunoglobulin variable region genes include coding and non-coding sequences for human heavy chain functional V_{H}, D_{H}, J_{H}, or coding and non-coding sequences for human light chain functional V_{L}, J_{L}, the light chain is a kappa or lambda light chain.

Further, the endogenous immunoglobulin variable region genes comprise mouse immunoglobulin heavy chain variable regions V_{H}, D_{H}, J_{H} or light chain variable regions V_{L}, J_{L}, wherein the light chain is a kappa or lambda light chain.

Further, the coding and non-coding sequences for the human heavy chain functional V_{H}, D_{H}, J_{H} are from human chromosome 14 and the coding and non-coding sequences for the human light chain functional V_{L}, J_{L} are from human chromosome 2 or 22.

Further, the coding and non-coding sequences for the human heavy chain functional V_{H}, D_{H}, J_{H} comprise sequences between nucleotide positions 105863198 and 106879844 from human chromosome 14, all coordinates refer to the GRCh38.p13 version of the human genome database from ENSEMBL, including one or more V_{H} region genes of the sequence number H1, H3, H5, H6, H7, H8, H10 (which fragment comprises 3 functional V region genes), H12, H14, H16, H18, H20, H22, H24, H25, H27, H29, H31, H33, H36, H38, H40, H42, H44, H45, H47, H48, H50, H52, H54, H55, H56, H58, H60, H62, H63, H65, H66, H67 fragments as described in Table 1, preferably 10-41 functional V_{H} region genes, more preferably 15-41 functional V_{H} region genes, more preferably 18-41 functional V_{H} region genes, more preferably 22-41 functional V_{H} region genes, more preferably 25-41 functional V_{H} region genes, for example 25, 26, 27, 28, 29, 30, 31, 32, 33, 34, 35, 36, 37, 38, 39, 40 or 41 functional V region genes.

Further, the coding and non-coding sequences for human light chain functional V_{L}, J_{L} comprise sequences between nucleotide positions 88860568 and 90235398 from human chromosome 2, including one or more V_{L} region genes of the sequence number K1, K2, K4, K5, K7, K8, K10, K11, K13, K14, K15, K17, K18, K20, K22, K23, K25, K27, K29, K30 fragments as described in Table 1; or sequences between nucleotide positions 22023114 and 22922913 from human chromosome 22, including one or more V_{L} region genes of the sequence number L1, L3, L5, L6, L7, L8, L9, L11, L13, L15, L16, L18, L20, L22, L24, L26, L27, L29, L31, L32, L33, L34, L35, L37, L39, L40, L42, L44, L46, L47, L49 fragments as described in Table 1, wherein all coordinates refer to the GRCh38.p13 version of the human genome database from ENSEMBL.

Further, the endogenous immunoglobulin variable region genes are partially or entirely deleted by homologous recombination, the human immunoglobulin heavy chain variable region genes are inserted at a location 3 KB upstream to 3 KB downstream from the deleted endogenous immunoglobulin heavy chain variable region, and the human immunoglobulin light chain variable region genes are inserted at a location 3 KB upstream to 3 KB downstream from the deleted endogenous immunoglobulin kappa light chain variable region.

The number of pseudogenes and/or open reading frame genes of the human immunoglobulin variable region genes knocked out (or, partially or entirely knocked out) should be sufficient such that the length of the various genes is shortened, and particularly sufficient to be shortened to a greater extent. Generally, the length of the human immunoglobulin heavy chain, Lambda light chain variable region genes inserted into the genome of the non-human mammalian cell is 10%-50%, preferably 12%-47%, preferably 14%-45%, preferably 15%-43%, more preferably 16%-40%, more preferably 16.10%, 18%, 18.50%, 20%, 25%, 30%, 31%, 31.75%, 35%, 38% or 38.06% of the total length of the human immunoglobulin heavy chain, Lambda light chain variable region genes, respectively, before the knockout of the pseudogenes and/or open reading frame genes. In addition, the length of the human immunoglobulin kappa light chain variable region genes inserted into the genome of the non-human mammalian cell is 35%-65%, preferably 37%-63%, preferably 38%-61%, preferably 40%-60%, preferably 42%-58%, preferably 45%-57%, preferably 47%-56%, more preferably 50%-55%, such as 51%, 52%, 53%, 53.08% or 54% of the total length of the human immunoglobulin kappa light chain variable region gene before the knockout of the pseudogenes and/or open reading frame genes.

The total number of pseudogenes and/or open reading frame genes is about 75 in the human heavy chain variable region, about 20 in the human kappa light chain proximal variable region, and about 42 in the human lambda light chain variable region.

Preferably, for “partially or entirely knocked out” or “partially or entirely deleted”, 10-100% (preferably 15-95%, 20-90%, e.g. 25%, 30%, 35%, 40%, 45%, 50%, 55%, 60%, 65%, 70%, 72%, 75%, 78%, 80%, 83%, 85% or 88%) of the pseudogenes and/or open reading frame genes are knocked out or deleted, the percentage is based on the total number of pseudogenes and open reading frame genes of the human immunoglobulin variable region genes.

Further, the non-human mammalian cell is a mouse embryonic stem cell, and the deleted endogenous immunoglobulin heavy chain variable region is located between positions 113428530 and 116027502 on mouse chromosome 12; the deleted endogenous immunoglobulin kappa light chain variable region is located between positions 67536984 to 70723924 on mouse chromosome 6; the deleted endogenous immunoglobulin lambda light chain variable region is located between positions 19065021 to 19260700 on mouse chromosome 16; wherein the mouse genome chromosomal location coordinates refer to the locations of the GRCm38.p6 version of C57BL/6J mouse genome database from ENSEMBL.

Preferably, the insertion site of the human immunoglobulin heavy chain variable region genes is position 113428513 on mouse genomic chromosome 12; the insertion site of the human immunoglobulin kappa light chain variable region genes is position 70723924 on mouse genomic chromosome 6; the insertion site of the human immunoglobulin lambda light chain variable region genes is position 70726758 on mouse genomic chromosome 6; the mouse genome chromosomal location coordinates refer to the locations of the GRCm38.p6 version of C57BL/6J mouse genome database from ENSEMBL.

Another aspect of the present invention provides a non-human mammalian cell comprising the genetically engineered recombinant genome. The mammalian cell is a non-human mammalian embryonic stem cell, more preferably, the embryonic stem cell is a mouse embryonic stem cell, a rat embryonic stem cell, or a rabbit embryonic stem cell.

The present invention also provides a recombinant cell comprising a nucleic acid construct that is prepared by introducing the nucleic acid construct of the present invention into a target cell. Optionally, the cell is an immortalized cell or a non-immortalized cell (e.g., a primary cell, a passaged cell), including a prokaryotic or eukaryotic cell, e.g., an E. coli cell, a yeast cell, an avian cell, a mammalian cell, a rat or mouse embryonic stem cell, an avian primordial germ cell, a C57BL/6J* 129S3 embryonic stem cell.

Also provided is the use of the nucleic acid construct, recombinant cell of the present invention in the preparation of a transgenic animal.

In another aspect, the present invention provides an engineered non-human mammalian cell, in the immunoglobulin loci of the genome thereof, immunoglobulin variable region genes endogenous to the host non-human mammalian cell are deleted, and gene segments having both coding and non-coding regions derived from human immunoglobulin variable regions are inserted; the pseudo-V-genes and/or open reading frames of the gene fragments of the human immunoglobulin variable region are partially or entirely deleted.

Typically, the cell is an immortalized cell or a non-immortalized cell (e.g., a primary cell or a passaged cell).

In the engineered non-human mammalian cell of the present invention, the inserted gene segments of the human immunoglobulin variable region comprise heavy chain variable region functional V, D, J region coding sequences and non-coding sequences, and/or, coding sequences and non-coding sequences for κ or λ light chain variable region functional V, J regions. Optionally, the non-human mammal is an avian, rodent, etc., for example, a mouse, rat, chicken, rabbit, etc., and the non-human mammal cell may be from an avian, rodent, mouse, rat, chicken, rabbit, etc., may be a rat or mouse embryonic stem cell, an avian primordial germ cell, a C57BL/6J* 129S3 embryonic stem cell, etc. Alternatively, the variable region gene segments of the endogenous immunoglobulin loci that are deleted include mouse immunoglobulin heavy chain variable region V, D, J regions or light chain κ or λ variable region V, J regions. Optionally, coding and non-coding sequences for functional V, D, J regions of the heavy chain variable regions inserted comprise sequences between nucleotide positions 105863198 and 106879844 from human chromosome 14; optionally, coding and non-coding sequences for κ light chain variable region functional V, J regions inserted comprise sequences between nucleotide positions 88860568 and 90235398 from human chromosome 2; optionally, coding sequences and non-coding sequences for the λ light chain variable region functional V, J region inserted comprise sequences between nucleotide positions 22023114 and 22922913 from human chromosome 22; and, all nucleotide position coordinates refer to the GRCh38.p13 version of the human genome database from ENSEMBL.

Another aspect of the present invention provides a method of producing a non-human mammalian cell, comprising:
a) introducing identical orientated and compatible recombinase targeting sites to upstream and downstream respectively of the variable region clusters of the immunoglobulin gene locus in a host non-human mammalian cell genome; b) continuing to introduce a specific recombinase capable of recognizing the recombinase targeting sites into the cell of step a), partially or entirely deleting the variable region genes of the immunoglobulin loci endogenous to the host non-human mammalian cell under the conditions allowing recombination to take place between the two recombinase targeting sites of step a), resulting in targeting cells; c) providing a vector comprising part or all of the variable regions of human immunoglobulin loci, knocking out part or all of the pseudo-V-genes and/or open reading frames in the variable region genes of the human immunoglobulin loci comprised in the vector, resulting in a targeting vector; d) introducing the targeting vector into the targeting cell obtained in step b) such that the variable regions of the human immunoglobulin loci comprised in the targeting vector replace the variable region genes of the endogenous immunoglobulin loci deleted in the targeting cell, thereby obtaining the engineered non-human mammalian cell.

Optionally, the targeting vector of step c) is a heavy chain targeting vector comprising a heavy chain variable region gene or a light chain targeting vector comprising a κ or λ light chain variable region gene. In addition, constructing one or more heavy chain targeting vectors or light chain targeting vectors may be constructed according to the number of variable region genes that need to be introduced, such as the targeting vectors shown in FIGS. 10, 13, and 14. When the heavy or light chain targeting vectors are plural, they may be introduced sequentially in step d).

Constructing the targeting vector of step c) may be conducted in E. coli or yeast cells.

More specifically, another aspect of the present invention provides a method of producing a non-human mammalian cell, the method comprising:
a) Deleting or inactivating the light and heavy chain variable regions of a host non-human mammal Ig locus; b) inserting a human IgH VDJ region that part or all of the pseudo-V-genes and/or open reading frame genes are deleted upstream of the heavy chain constant region of the Ig locus of the host non-human mammal, the human IgH VDJ region comprising a plurality of human IgH V regions, one or more human D regions, and one or more human J regions, and/or; c) inserting a human κ VJ region that part or all of the pseudo-V-genes and/or open reading frames are deleted upstream of a κ constant region of the host non-human mammal Ig locus, the human κ VJ region comprising a plurality of human Ig light chain κ V regions and one or more human Ig light chain κ J regions, and/or; d) inserting a human λ VJ region that part or all of the pseudo-V-genes and/or open reading frames are deleted downstream of a κ constant region of the host non-human mammal Ig locus, the human λ VJ region comprising a plurality of human Ig light chain λ V regions and one or more human Ig light chain λ J regions; wherein steps a)-c) can be carried out in any order and can be carried out stepwisely or in parallel.

In addition, another aspect of the present invention provides a method of producing a non-human mammalian cell, the method comprising:
a) introducing identical orientated and compatible recombinase targeting sites to upstream and downstream respectively of the immunoglobulin variable region gene in the genome of a non-human mammalian cell;
b) introducing a specific recombinase capable of recognizing the targeting sites of step a), allowing recombination event to occur between the two recombinase targeting sites of step a) resulting in partial or entire deletion of the endogenous immunoglobulin variable region genes of the non-human mammalian cell;
c) providing a targeting vector comprising part or all of the human immunoglobulin variable region, wherein the targeting vector contains human functional variable region genes and part or all of the pseudogenes and/or open reading frames are knocked out; the human functional variable region genes comprise coding and non-coding sequences for human heavy chain functional V_{H}, D_{H}, J_{H}, or coding and non-coding sequences for human light chain functional V_{L}, J_{L}, the light chain is a kappa or lambda light chain; and the targeting vector is selected from BAC vector or YAC vector;
d) introducing the targeting vector of step c), resulting in the replacement of the deleted non-human mammalian cell endogenous immunoglobulin gene of step b) by the human immunoglobulin variable region gene in step c) in the non-human mammalian cell;
e) generating the non-human mammalian cell comprising human immunoglobulin variable region genes in the genome from step d).

Preferably, the targeting vector of step c) is constructed in E. coli or yeast cells.

Another aspect of the present invention provides a targeting vector, comprising human immunoglobulin variable region genes, a part or all of the pseudogenes and/or open reading frames of the human immunoglobulin variable region genes are knocked out, wherein the human immunoglobulin variable region genes comprise coding and non-coding sequences for human heavy chain functional V_{H}, D_{H}, J_{H}, or coding and non-coding sequences for human light chain functional V_{L}, J_{L}, the light chain is a kappa or lambda light chain. The targeting vector is selected from a BAC vector or a YAC vector.

Another aspect of the invention provides a method of generating a non-human mammal expressing an antibody that is fully human in variable regions, introducing the non-human mammalian cell into the utero of a female wild-type non-human mammal, selecting the progeny chimeric non-human mammal as F0 generation non-human mammal.

Further, prior to introducing the non-human mammal cells into the utero of a female wild-type non-human mammal, the non-human mammalian cells are screened to obtain a non-human mammalian cell clone having no increase or decrease in chromosome number, the non-human mammalian cell clone is transplanted into a wild-type non-human mammalian embryonic blastocoel, and the blastocyst is transplanted into a pseudopregnant female wild-type non-human mammalian utero.

Further, the F0 generation non-human mammal is propagated with a wild-type non-human mammal to obtain a stably inheritable F1 generation non-human mammal having human immunoglobulin variable region genes inserted at specified positions. Further, a non-human mammal expressing an antibody having both a fully human heavy chain variable region and a fully human light chain variable region is obtained by breeding a non-human mammal expressing an antibody having a fully human heavy chain variable region with a non-human mammal expressing an antibody having a fully human light chain variable region as parents.

Another aspect of the present invention provides a non-human mammal prepared by the method of generating a non-human mammal expressing an antibody that is fully human in variable region.

Preferably, the non-human mammal is a mouse, a rat, or a rabbit, and the non-human mammalian cell is a mouse embryonic stem cell, a rat embryonic stem cell, or a rabbit embryonic stem cell.

Another aspect of the present invention provides the use of the recombinant genome, the non-human mammalian cell, the targeting vector or the obtained non-human mammal in screening an antibody with fully human variable regions or in the process of preparing fully human antibody drugs.

Another aspect of the present invention provides the use of the recombinant genome, the non-human mammalian cell, the method of producing a non-human mammalian cell, or the targeting vector in preparation of a non-human mammal.

Another aspect of the present invention provides an antibody or an antibody fragment with fully human variable region produced by the non-human mammal, or a derivative drug or pharmaceutical composition comprising the antibody or antibody fragment.

The present invention also provides the use of an engineered non-human mammalian cell in preparing a transgenic animal and a method of producing a transgenic animal, the method comprises: injecting engineered non-human mammalian cells of the present invention, such as embryonic stem cells, into blastocysts, followed by implantation of the chimeric blastocysts into females to produce offsprings, and propagating and selecting homozygous recombinants with desired insertions to obtain transgenic animals. Optionally the animal is an avian, rodent, etc., and can be a rat, mouse, chicken, or rabbit.

In another aspect, the present invention provides a method of producing an antibody or antigen-binding fragment thereof, comprising immunizing the transgenic animal produced according to the present invention with an antigen, and recovering the antibody or antibody chain or recovering cells producing the antibody or heavy or light chain. Optionally, the constant region of the resulting antibody or antigen-binding fragment thereof is replaced with a human constant region to generate a fully humanized antibody. The present invention also provides antibodies or antigen-binding fragments thereof prepared, and their use in the preparation of pharmaceutical compositions, as well as pharmaceutical compositions comprising these antibodies or antigen-binding fragments thereof, optionally further comprising a pharmaceutically acceptable carrier; the pharmaceutical composition can also be an antibody-derived drug comprising an antibody conjugated to other molecules, such as an antibody small molecule toxin conjugates, an antibody radioimmune conjugates, an antibody therapeutic polypeptide conjugates, a bi/multispecific antibody, and the like.

All coordinates of human immunoglobulin genes in the present invention refer to the version GRCh38.p13 of the human genome database from ENSEMBL, human heavy chain variable region DNA fragment is derived from the part between nucleotide positions 105863198 and 106879844 of human chromosome 14, human Kappa light chain variable region DNA fragment is derived from the part between positions 88860568 and 90235398 of human chromosome 2, human Lambda light chain variable region DNA fragment is derived from the part between positions 22023114 and 22922913 of human chromosome 22.

Of the total number of pseudo-V-genes and open reading frames in the variable region gene segments of a human immunoglobulin locus, typically 10-100% of the pseudo-V-genes and/or open reading frames, for example 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99% or 100%, are knocked out.

The method of gene knockout in the present invention can be any suitable method known in the art, e.g. knockout using homologous recombination, for illustrative examples see the Figures.

The recombinase in the present invention can be any suitable enzyme known in the art, such as Cre, FLP and the like, and the recognition site may be LoxP, FRT and the like. Combinations of homologous recombination and site-specific recombination can be utilized to create the construct, cell, and animal of the present invention. Exemplary homologous recombination methods are described in U.S. Pat. Nos. 6,689,610, 6,204,061, 5,631,153, 5,627,059, 5,487,992, and 5,464,764, which are incorporated herein by reference. Site-specific recombination requires dedicated recombinases to recognize sites and catalyze recombination at these sites. Many bacteriophage and yeast-derived site-specific recombination systems, such as the bacteriophage PI Cre/LoxP of tyrosine family, the yeast FLP-FRT system, and the Dre system, each including a recombinase and specific homologous sites, are useful for integration of DNA in eukaryotic cells and are also suitable for the present invention. Such systems and methods of use are described, for example, in U.S. Pat. Nos. 7,422,889, 7,112,715, 6,956,146, 6,774,279, 5,677,177, 5,885,836, 5,654,182, and 4,959,317, which are incorporated herein by reference. The recombinase-mediated cassette exchange (RMCE) procedure is performed by using a combination of wild-type and mutated LoxP (or FRT, etc.) sites along with negative selection. Other systems of the tyrosine family, such as bacteriophage λ Int integrase, HK2022 integrase, and other systems belonging to the serine family of recombinases, such as bacteriophage phiC31, R4Tp901 integrase, are also suitable for the present invention. Introduction of site-specific recombination sites can be achieved by conventional homologous recombination techniques which are described in references such as Sambrook and Russell (2001) (Molecular cloning: a laboratory manual, 3rd Edition (Cold Spring Harbor, Nundefined Y.: Cold Spring Harbor Laboratory Press) and Nagy, A. (2003). (Manipulating the mouse embryo: a laboratory manual, 3rd Edition (Cold Spring Harbor, N.Y.: Cold Spring Harbor Laboratory Press). Genetic Recombination: Nucleic acid, Homology (biology), Homologous recombination, Non-homologous end joining, DNA repair, Bacteria, Eukaryote, Meiosis, Adaptive immune system, V (D) J recombination by Frederic P. Miller, Agnes F. Vandome and John McBrewster (Paperback-Dec. 23, 2009).

The gene knocked-out or knocked-in can be identified using any method known in the art, including but not limited to, enzyme cleavage identification, PCR identification, hybridization or screening markers (e.g., resistance, nutrition, toxin selection, etc.) identification, exemplary means are shown in FIGS. 11, 12.

The targeting vector used in the present invention may be any known type suitable for the present invention. A typical gene targeting vector generally consists of three parts, namely containing a gene for targeting or an exogenous gene to be inserted into the genome of a recipient cell, DNA sequences homologous to the target locus within the cell flanking the exogenous gene, and a marker for screening. Usually the neomycin phosphotransferase gene (neo) is used as a positive (+) selection marker, and recipient cells expressing the neomycin phosphotransferase gene can be screened by culturing on G418-containing medium. Exemplary targeting vector is BAC vector.

Recombineering methods for producing vectors for homologous recombination in cells in the present invention are described, for example, in WO9929837 and WO0104288, such techniques are well known in the art. In one aspect, recombineering of human DNA is performed using BAC as a source of human DNA. Human BAC DNA is isolated using the MN NucleoBond BAC 100 Purification Kit. The genomic insert of each human BAC is edited using recombineering, whereby once inserted, a seamless contiguous portion of the human V (D) J genomic region is formed at the mouse IgH or IgK locus. Electroporation transfection and genotyping of BAC DNA can refer to standard protocols (Prosser, Hundefined M., Rzadzinska, A. K., Steel, K. P., and Bradley, A. (2008). Mosaic complementation demonstrates a regulatory role for myosin Vila in actin dynamics of stereocilia. Molecular and Cellular Biology 28, 1702-1712; ramirez-Solis, R., Davis, A. C., and Bradley, A. (1993). Gene targeting in embryonic stemcells. Methods in Enzymology 225, 855-878.).

The engineered non-human mammalian cell of the present invention can be used to generate transgenic animal, thereby producing antibodies or antigen-binding fragments thereof comprising human immunoglobulin variable regions. In one aspect, the host cell into which the endogenous immunoglobulin gene is replaced is an embryonic stem cell that can then be used to produce a transgenic mammal. Thus, the method of the present invention further comprises isolating embryonic stem cells comprising introduced portions of human immunoglobulin variable regions and using the embryonic stem cells to produce transgenic animals comprising partially replaced immunoglobulin loci. Optionally, the transgenic animal may be avian, and the transgenic animal is produced using primordial germ cells. Thus, the method of the present invention further comprises isolating primordial germ cells comprising introduced portions of the human immunoglobulin variable regions and using the germ cells to produce transgenic animals comprising partially replaced immunoglobulin loci. Methods for producing such transgenic avian are disclosed, for example, in U.S. Pat. Nos. 7,323,618 and 7,145,057, which are incorporated herein by reference.

Transgenic animals of the present invention can be used to produce human antibodies, e.g., polyclonal antibodies and monoclonal antibodies. These antibodies may be used for conventional uses in the art, including various purposes of preparing compositions, such as pharmaceutical compositions, detecting antigens, such as detecting reagents or kits, or diagnostics, such as diagnostic reagents or kits, etc. Antigen immunization and methods of preparing antibodies as well as techniques for preparing compositions, products for detection or diagnosis are all well known in the art.

### Advantageous Effects of the Invention

1) By deleting part or all of the pseudogenes and/or open reading frames, functional human antibody variable region gene fragments are included as many as possible in the same vector to achieve highly efficient V_{H} D_{H} J_{H} or VOL recombination in mice.
2) More functional human antibody gene fragments are introduced into experimental animals with smaller vectors, resulting in reduction of construction risk, construction time, and construction cost.
3) Such knock-outs are highly efficient and less time-consuming since knocking out pseudogenes or open reading frames of the present invention are done in E. coli with ease of operation; by pruning human immunoglobulin variable region genes in E. coli, the steps of gene targeting in embryonic stem cells will be minimized, even it can be accomplished in one step, thus, only few non-human mammalian cell gene targeting steps are required to accomplish as many functional human antibody variable region genes introduction as possible, and a laboratory animal expressing fully human variable regions can be constructed in a short time.
4) Experimental animals constructed by the present invention can normally express chimeric antibodies with variable regions of fully human origin and constant regions of murine origin, which also have a similar or higher level of specific immune response to antigen than wild-type mice.
5) Compared to existing antibody screening platforms, the transgenic mice of the present invention can generate larger antibody repertoires that can be used to efficiently screen antibodies, resulting in antibodies with higher affinities in nM level, even in pM.
6) The ratio of mature B cells and immature B cells in the spleen of the transgenic mice of the present invention is indistinguishable from wild-type mice, ensuring highly efficient and normal B cell development.

### DESCRIPTION OF THE DRAWINGS

FIG. 1: Schematic representation of the segmental insertion of DNA fragments derived from the genome of human immunoglobulin heavy chain, Kappa and Lambda light chain loci into BAC or YAC vectors, and the basic structure of functional variable region V gene segments, pseudo variable region V gene segments and open reading frame V gene segments;

FIG. 2: Schematic of recombination knockout when two recombinase recognition sites are located on the same chromosome and in the same orientation;

FIG. 3: Schematic representation of translocation events when two recombinase recognition sites are located on two homologous chromosomes and in the same orientation;

FIG. 4: Schematic representation of the method for efficient knockout of long target fragment DNA sequences between recombinase recognition sites by recombination to combine into complete antibiotic screening gene expression elements, after introduction of compatible recombinase recognition sites in the same orientation and antibiotic screening gene expression elements divided in two halves to the same chromosome in the genome step by step;

FIG. 5: Schematic representation of recombinant knockout of mouse genome endogenous immunoglobulin heavy chain variable region (sequence between 113428530 to 116027502 on mouse chromosome 12) (PolyA-hygromycin-LoxP, Puro-CAG, PolyA-Neo-LoxP-PGK, PolyA-hygro-LoxP-PGK);

FIG. 6: Schematic representation of recombinant knockout of mouse genome endogenous immunoglobulin kappa light chain variable region (sequence between 67536984 to 70723924 on mouse chromosome 6);

FIG. 7: Schematic representation of recombinant knockout of mouse genome endogenous immunoglobulin lambda light chain variable region (sequence between 19065021 to 19260700 on mouse chromosome 16);

FIG. 8: The flowchart for knockout of a pseudogene or open reading frame in a BAC vector comprising DNA fragments of human immunoglobulin region;

FIG. 9: Schematic representation of reassembly of 5′ end gene regulatory regions, V region coding sequences (including introns and exons) and 3′ antibody gene recombination signal sequence (RSS) of different functional V region genes into a completely new functional V region gene fragment;

FIG. 10: Schematic diagram of the sequence and structure of two targeting vectors comprising human heavy chain immunoglobulin variable region gene fragments;

FIG. 11: schematic illustration of a method for identifying the integrity of DNA fragment of the human immunoglobulin region contained in the BAC vector, taking an example of a bacterial artificial chromosome enzyme digestion pulsed electrophoresis gel imaging of heavy chain targeting vector 1;

FIG. 12: schematic illustration the method of identifying the integrity of the human immunoglobulin gene fragment by PCR method, using the bacterial artificial chromosome of the heavy chain targeting vector 1 as a PCR template;

FIG. 13: Schematic diagram of sequence and structure of targeting vector comprising human Kappa light chain immunoglobulin variable region gene fragments;

FIG. 14: Schematic diagram of sequences and structures of two targeting vector comprising human Lambda light chain immunoglobulin variable region gene fragments

FIG. 15: Schematic diagram for insertion of human immunoglobulin heavy chain variable region genes into mouse endogenous immunoglobulin heavy chain variable region location (original 113428530 to 116027502 on chromosome 12 are deleted);

FIG. 16: Schematic diagram of staged introduction of human heavy chain targeting vectors 1 and 2 into mouse embryonic stem cells lacking mouse endogenous heavy chain variable region sequences;

FIG. 17: Schematic diagram for illustrating PCR identification of accurate gene insertion events in the protocol for accurate site-directed insertion of gene fragments by homologous recombination using ACE001-H2 single homology arm targeting vectors in FIG. 2 as an example;

FIG. 18: schematic illustration of the method and electrophoresis results for 5′ and 3′ PCR using primers P1/P4 and P3/P2 to identify accurate gene insertion events, using ACE001-H2 single homology arm targeting vector in FIG. 2 as an example;

FIG. 19: Schematic diagram for insertion of human immunoglobulin Kappa light chain variable region gene into the location of mouse endogenous immunoglobulin Kappa light chain variable region (original 67536984 to 70723924 of chromosome 6 deleted);

FIG. 20: Schematic diagram of the introduction of a human kappa light chain targeting vector into a mouse embryonic stem cell deleted of mouse endogenous kappa light chain variable region sequence;

FIG. 21: Schematic diagram for insertion of the human immunoglobulin Lambda light chain variable region gene into the position downstream of the mouse endogenous immunoglobulin Kappa light chain constant region (original 67536984 to 70723924 of chromosome 6 deleted);

FIG. 22: Schematic diagram of the staged introduction of human Lambda light chain targeting vectors 1 and 2 into mouse embryonic stem cells lacking the mouse endogenous kappa light chain sequence;

FIG. 23: Comparison of B cell development in transgenic mice of the present invention versus wild-type BALB/c mice;

FIG. 24: Comparison of OVA-specific serum antibody levels post the third booster immunization in transgenic versus wild-type BALB/c mice;

FIG. 25: Affinity level of antibodies obtained from transgenic mice of the present invention.

FIG. 26: SEQ ID NO. 1 in step 2 of Example 4.

### Definitions

Pseudogene: is a nonfunctional residue formed by a gene family during evolution. A pseudogene can be considered as a non-functional copy of genomic DNA in the genome that closely resembles the coding gene sequence, which is not generally transcribed and has no clear physiological significance. Pseudogenes have homologous normal genes, and their DNA sequences are very similar. The ancestor genes of pseudogenes are functional but disabled due to the failure to be transcribed resulting from mutation, or their transcription products cannot be translated. Pseudogenes are ubiquitous in mammalian genomes and can be considered as relics of evolution.

Open reading frame (ORF): An open reading frame is a base sequence fragment of mRNA, starting at a start codon and ending at a stop codon, and an ORF corresponds to a protein.

For specific definitions and characteristics of pseudogenes and open reading frames in human or mouse immunoglobulin loci, refer to the following illustrative link of IMGT database: http://www.imgt.org/IMGTScientificChart/SequenceDescription/IMFTfunctionality.html.

Immunoglobulin heavy chain variable region (V_{H}): the region of an immunoglobulin heavy chain molecule where the amino acid sequence varies broadly. A functional region of about 115-120 residues from the amino terminus. Among them, there are three hypervariable regions with more significant changes, the amino acid residues thereof are located at positions 29-31, 49-58, and 95-102, respectively.

Immunoglobulin light chain variable region (V_{L}): The region of an immunoglobulin light chain molecule where the amino acid sequence varies widely. It contains a functional region of about 110 amino acid residues. Among them, there are three portions that vary significantly, called hypervariable regions, the amino acid residues thereof are positions 28-35, 49-56, and 91-98.

Immunoglobulin gene rearrangement: As B lymphocytes differentiate, immunoglobulin genes can undergo rearrangement phenomena such as V_{H}/D_{H}/J_{H}, V_{L}/J_{L}, and the like, resulting in diversity of immunoglobulins.

Coding sequence: is a base sequence of DNA that encodes the mature RNA base sequence within the transcribed region, e.g., an exon. Only less than 2% of human genome sequences are coding sequences.

Non-coding sequence: \textcircled{1} All sequences in the gene sequence other than the coding sequence, e.g., promoter, intron, and enhancer. \textcircled{2} All sequences in the genomic sequence other than the coding sequence of the gene. More than 98% of human genomic sequences are non-coding sequences.

Targeting vector: A typical gene targeting vector generally consists of three parts, i.e., containing a gene for targeting or an exogenous gene to be inserted into the genome of a recipient cell, DNA sequences homologous to the target locus within the cell on one or both flanks of the exogenous gene, and a marker for screening. Usually the neomycin phosphotransferase gene (neo) is used as a positive (+) selection marker, and recipient cells expressing the neomycin phosphotransferase gene can be screened by culturing on G418-containing medium.

Derivatized drug comprising an antibody or antibody fragment: a drug comprising an antibody or antibody fragment and conjugated to other molecules, such as an antibody small molecule toxin conjugates, an antibody radioimmune conjugates, an antibody therapeutic polypeptide conjugates, a bi/multispecific antibody, and the like.

### DETAILED DESCRIPTION

Before the present invention is further described, it is to be understood that the present invention is not limited to the particular embodiments described therein, since routine variations to the elements of such embodiments may be made by those skilled in the art using known techniques.

Unless defined otherwise, all technical and scientific terms used herein have the same meaning as commonly understood by the person skilled in the art. All publications mentioned herein are incorporated herein by reference to disclose and describe the methods and/or materials relating to the cited publications.

In the following examples, all mouse genomic chromosomal location coordinates refer to the locations of the version GRCm38.p6 of C57BL/6J mouse genome database from ENSEMBL and all human genomic chromosomal location coordinates refer to the version GRCh38.p13 of the human genome database from ENSEMBL.

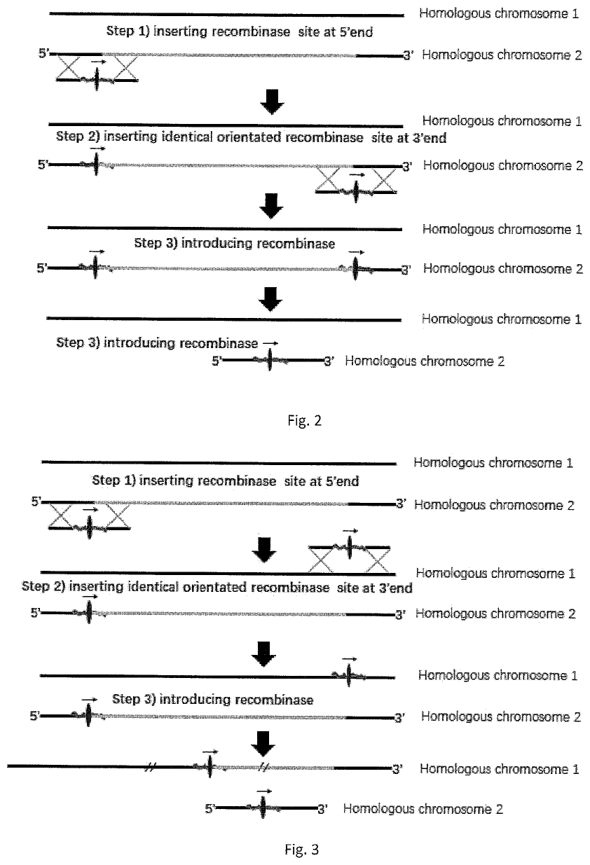

The stepwise knockout method of DNA fragments of the present invention, as shown in FIG. 2, knocks out a fragment of interest in a cell by several steps, comprising: 1) inserting a DNA fragment containing a recombinase recognition site (e.g., LoxP or FRT) at the 5 ′end of the fragment of interest in the cell by homologous recombination; 2) inserting a DNA fragment containing a recombinase recognition site (e.g., LoxP or FRT) compatible with and in the same orientation as the recombinase recognition site of step 1) at the 3 ′end of the fragment of interest of the same cell clone completed in step 1) by homologous recombination; 3) introducing a recombinase (e.g. Cre or FLP) recognizing the recombinase recognition site inserted in step 1) or 2) into the same cell clone completed in step 2), and when both recombinase recognition sites are located on the same DNA and in the same orientation, the recombinase can efficiently excise the sequence between the two recombinase sites to achieve the knockout of the fragment of interest (as shown in FIG. 2).

In diploid cells, since the fragment of interest is present on two different chromosomes, homologous recombination-mediated recombinase recognition site insertion in step 1) and step 2) described above will occur on two different chromosomes in 50 percent, in this case it is still possible for the recombinase recognition sites in the same orientation to be recognized by the introduced recombinase in step 3), and translocation between homologous chromosomes takes place, as shown in FIG. 3, in which case knock-out of the fragment of interest can still be achieved.

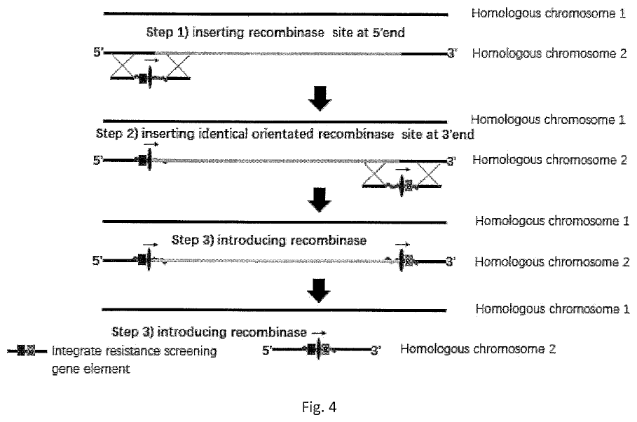

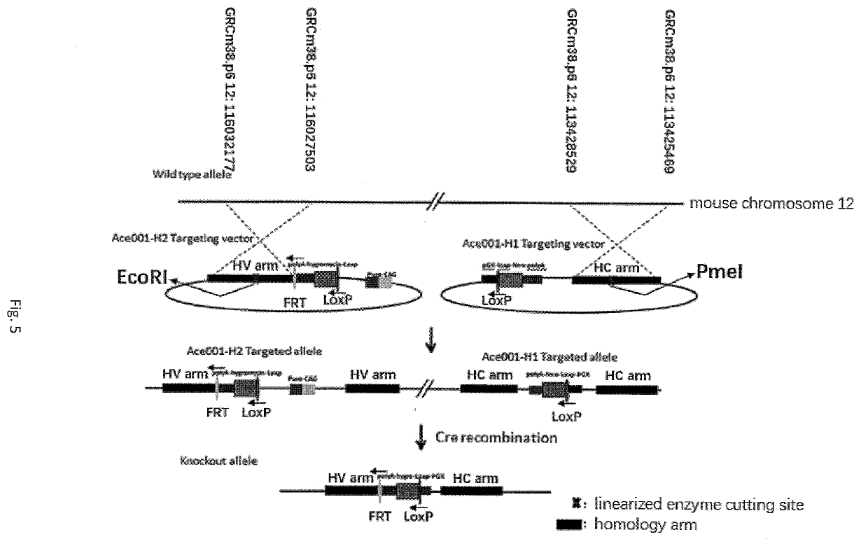

The longer the fragment of interest, the lower the efficiency of knocking out the fragment of interest by the method of step 1) to step 3), in this case, preferably, the same resistance screening gene (including promoter, coding region and poly-A transcription termination region) are divided into two parts A, B that have no resistance screening function, respectively, and carried into 5 ′and 3′ ends of recombinase recognition sites inserted to both ends of the gene of interest by steps 1) and 2) respectively, after the recombination event has occurred, the fragment of interest between the two recombinase sites is efficiently excised and the two parts A and B are recombined into a screening-functional resistance screening gene, resulting in efficient screening of cell clones that effectively knock out the gene fragment of interest (FIG. 4).

Mouse embryonic stem cells for gene knockout or human immunoglobulin variable region knockin can be derived from the strain such as 129, c57BL/6J, C57BL/6N, etc. or hybrid F1 generations, e.g. C57BL/6J*129 strain mouse embryonic stem cells, and such stem cells can be isolated from early mouse embryonic inner cell mass (Ref: Evans M. J., Kaufman M. H. (1981). Establishment in culture of pluripotent cells from mouse embryos. Nature 292, 154-156. 10.1038/292154 a0), or purchased from commercial providers, e.g., Cyagen (Cat. No. MUAES-01001 or MUBES-01001) or Applied Stemcell (Cat. No. ASE-9005, ASE-9006, ASE-9007, ASE-9008 or ASE-9005).

### Example 1: Knockout of Mouse Endogenous Heavy Chain Immunoglobulin Variable Region Locus

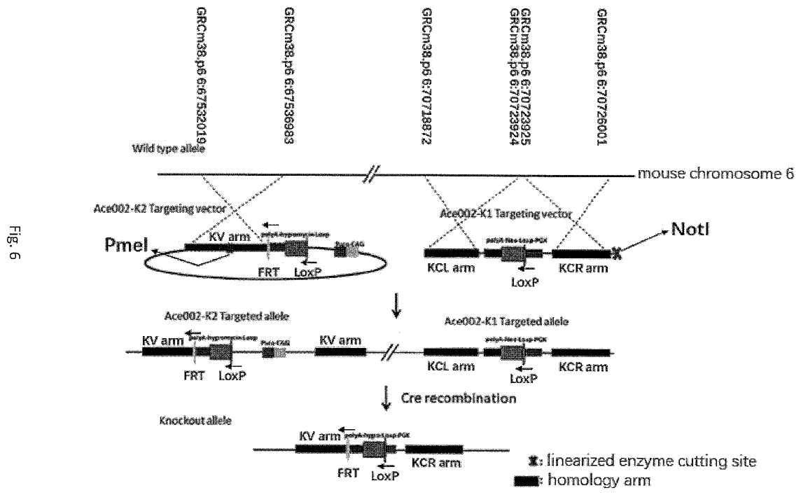

The overall strategy for knockout of a mouse endogenous heavy chain immunoglobulin variable region locus can refer to FIG. 5. Specific steps are as follows:

Step 1, Constructing two targeting vectors Ace001-H1, Ace001-H2, the construction of which is familiar to those skilled in the art.

The Ace001-H1 vector is shown in FIG. 5 and has the following features: 1) it contains the sequence as homology arms between 113428529 and 113425469 of mouse chromosome 12 (HC arm) and one unique linearized enzyme cutting site EcoRI is inserted at position 113426998; 2) it contains a neomycin screening gene expression element PGK-neo-polyA, wherein a recombinase recognition site LoxP is inserted before ATG of the translation initiation codon of the neomycin Neo coding gene;

The Ace001-H2 vector is as shown in FIG. 5 and has the following features: 1) it contains the sequence between 116032177 and 116027503 of mouse chromosome 12 (HV arm) as homology arms and one unique linearized enzyme cutting site Pmel is inserted at position 116029758; 2) it contains a puromycin resistance gene expression element CAG-puro-polyA with complete expression function; 3) it contains a hygromycin B resistance gene coding region without promoter and a polyA, and carries one recombinase recognition site FRT and LoxP at both ends of the element respectively; in Ace001-H1 and Ace001-H2 vectors, the orientation of FRT and LoxP is indicated with arrows.

Step 2, the Ace001-H1, Ace001-H2 vector were sequentially introduced into mouse embryonic stem cells, surviving embryonic stem cell clones were obtained by screening with 225 μg/ml Neomycin(supplier: Invitrogen (Shanghai) Trade Ltd., Cat. No. 10131027) or 1.25 μg/ml Puromycin (supplier: Invitrogen (Shanghai) Trade Ltd., Cat. No. A1113803) respectively, the clones were detected using conventional PCR means to obtain the embryonic stem cell clones with both vectors knocked into the correct mouse embryonic stem cell genomic location; then a vector expressing Cre recombinase was introduced into these embryonic stem cell clones, surviving embryonic stem cell clones were obtained by screening with 50 μg/ml Hygromycin B(supplier: Invitrogen (Shanghai) Trade Ltd., Cat. No. 10687010), the clones were detected using conventional PCR means to obtain the embryonic stem cell clones with of cre recombinase-mediated knockout of large fragment, one of the chromosomes of these mouse embryonic stem cell clones have a deletion of the mouse endogenous sequence between the two positions of 113428530 to 116027502 on chromosome 12; and in this step, Ace001-H1 and Ace001-H2 may be sequentially introduced into mouse embryonic stem cells in any order. In this step, the PCR of forward and reverse primer outside of the homology arm regions was used for identification of homologous recombination targeting vector knock-in of mouse embryonic stem cells (as shown in FIG. 17), the design of the primers and the specific operational steps of the PCR experiment are familiar to those skilled in the art.

### Example 2: Knockout of Mouse Endogenous Kappa Light Chain Immunoglobulin Variable Region Locus

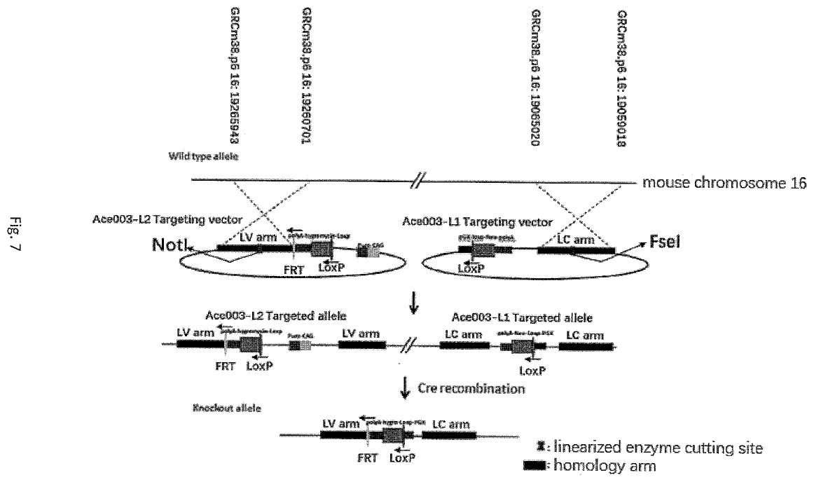

The overall strategy for knockout of the mouse endogenous Kappa light chain immunoglobulin variable region locus may refer to FIG. 6, comprising the following specific steps:

Step 1, Construction of two targeting vectors Ace002-K1, Ace002-K2.

The Ace002-K1 vector is as shown in FIG. 6 and has the following features: 1) it contains a sequence between 70718872 and 70723924 of mouse chromosome 6 (KCL arm) as left homology arm, a sequence between 70723925 and 70726001 of mouse chromosome 6 (KCR arm) as right homology arm, wherein downstream of KCR carries a unique linearized enzyme cutting site Notl; 2) a neomycin screening gene expression element PGK-neo-polyA is included between the left and right homology arms, wherein a recombinase recognition site LoxP is inserted before the translation initiation codon ATG of the neomycin Neo coding gene;

The Ace002-K2 vector is as shown in FIG. 6 and has the following features: 1) it contains the sequence between 67532019 and 67536983 of mouse chromosome 6 as homology arms and a unique linearized enzyme cutting site Pmel is inserted at position 67534443; 2) it contains one puromycin resistance gene expression element CAG-puro-polyA with complete expression function; 3) it contains a hygromycin B resistance gene coding region without promoter and a polyA, and carries one recombinase recognition site FRT and LoxP at both ends of the element respectively; in Ace002-K1 and Ace002-K2 vectors, the orientation of FRT and LoxP is indicated by arrows.

Step 2, Ace002-K1, Ace002-K2 vectors were sequentially introduced into mouse embryonic stem cells, surviving embryonic stem cell clones were obtained by screening with 225 μg/ml neomycin or 1.25 μg/ml Puromycin, respectively, the clones were detected using conventional PCR means to obtain embryonic stem cell clones with both vectors knocked into the correct mouse embryonic stem cell genomic location; then a vector expressing Cre recombinase was introduced into these embryonic stem cell clones, surviving embryonic stem cell clones were obtained by screening with 50 μg/ml Hygromycin B, these clones were detected using conventional PCR means to obtain embryonic stem cell clones with cre recombinase-mediated knockout of large fragment, one of the chromosomes of these mouse embryonic stem cell clones have a deletion of the mouse endogenous sequence between the two positions of 67536984 to 70723924 on chromosome 6; in this step, Ace002-K1 and Ace002-K2 may be sequentially introduced into mouse embryonic stem cells in any order. In this step, the PCR of forward and reverse primer outside of the homology arm regions was used for identification of homologous recombination targeting vector knock-in of mouse embryonic stem cells (as shown in FIG. 17), the design of the primers and the specific operational steps of the PCR experiment are familiar to those skilled in the art.

### Example 3: Knockout of Mouse Endogenous Lambda Light Chain Immunoglobulin Variable Region Locus

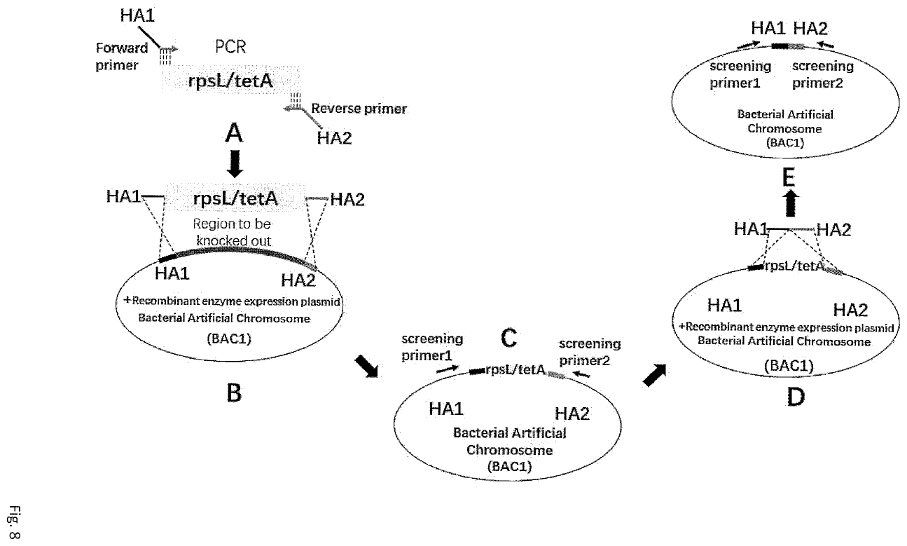

The overall strategy for knockout of the mouse endogenous lambda light chain immunoglobulin locus may refer to FIG. 7. Specific steps are described as:

Step 1, Construction of two targeting vectors Ace003-L1, Ace003-L2, the construction thereof is familiar to those skilled in the art.

The Ace003-L1 vector is as shown in FIG. 7 and has the following features: 1) it contains the sequence between 19065020 and 19059018 of mouse chromosome 16 (LC arm) as homology arms and one unique linearized enzyme cutting site Fsel is inserted at position 19061523; 2) it contains a neomycin screening gene expression element PGK-neo-polyA, wherein a recombinase recognition site LoxP is inserted before ATG of the translation initiation codon of the neomycin Neo coding gene;

The Ace003-L2 vector is as shown in FIG. 7 and has the following features: 1) it contains the sequence between 19265943 and 19260701 of mouse chromosome 16 (LV arm) as homology arms and one unique linearized enzyme cutting site Notl is inserted at position 19263393; 2) it contains one puromycin resistance gene expression element CAG-puro-polyA with complete expression function; 3) it contains a hygromycin B resistance gene coding region without promoter and a polyA, and carries one recombinase recognition site FRT and LoxP at both ends of the element, respectively; in Ace003-L1 and Ace003-L2 vectors, the orientation of FRT and LoxP is indicated by arrows.

Step 2, Ace003-L1, Ace003-L2 vectors were sequentially introduced into mouse embryonic stem cells, surviving embryonic stem cell clones were obtained by screening with 225 μg/ml neomycin or 1.25 μg/ml Puromycin, respectively, the clones were detected using conventional PCR means to obtain embryonic stem cell clones with both vectors knocked into the correct mouse embryonic stem cell genomic location; then a vector expressing Cre recombinase was introduced into these embryonic stem cell clones, surviving embryonic stem cell clones were obtained by screening with 50 μg/ml Hygromycin B, the clones were detected using conventional PCR means to obtain embryonic stem cell clones with Cre recombinase-mediated knockout of large fragment, one of the chromosomes of these mouse embryonic stem cell clones have a deletion of the mouse endogenous sequence between the two positions of 19065021 to 19260700 on chromosome 16; in this step, Ace003-L1 and Ace003-L2 may be sequentially introduced into mouse embryonic stem cells in any order. In this step, the PCR of forward and reverse primer outside of the homology arm regions was used for identification of homologous recombination targeting vector knock-in of mouse embryonic stem cells (as shown in FIG. 17), the design of the primers and the specific operational steps of the PCR experiment are familiar to those skilled in the art.

### Example 4: Knockout and Identification of Pseudogenes and Open Reading Frames in Human Immunoglobulin Variable Regions

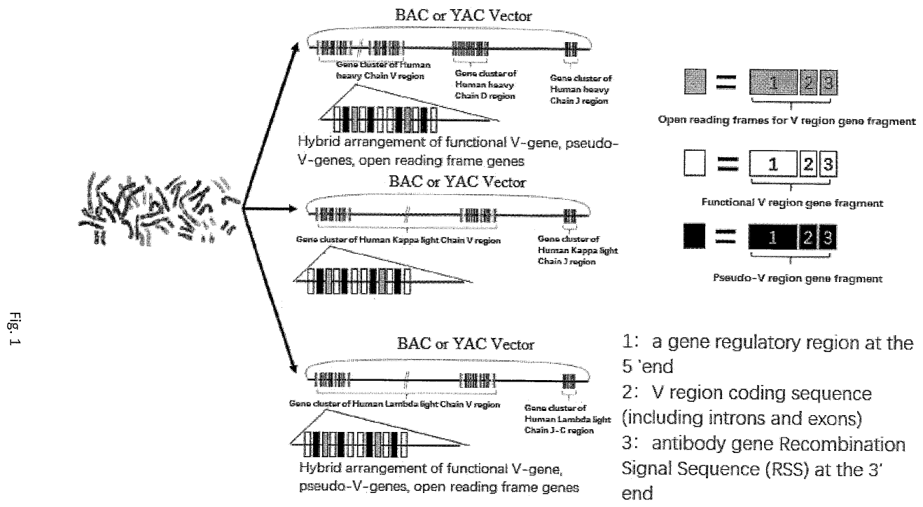

Human heavy chain variable region DNA fragment is derived between positions 105863198 and 106879844 of chromosome 14, human Kappa light chain variable region DNA fragment is derived between positions 88860568 and 90235398 of chromosome 2, human Lambda light chain variable region DNA fragment is derived between positions 22023114 and 22922913 of chromosome 22. As shown in FIG. 1, these DNA fragments derived from the human genome are respectively inserted into vectors containing non-human DNA fragments such as a bacterial artificial chromosome (BAC), a suitable BAC can be inquired by ENSEMBL, and the BAC vectors used in the present invention are purchased from the suppliers Source BioScience or Invitrogen (Shanghai) Trade Co., Ltd.

The process of removing unwanted pseudogene and open reading frame DNA fragments from a BAC comprising original human immunoglobulin variable region gene fragments is done in E. coli and an exemplary knockout process is shown in FIG. 8 and the specific steps are depicted as:

step 1, 1.1) A bacterial artificial chromosome BAC1 (carrying chloramphenicol resistance) comprising human immunoglobulin variable region DNA fragments is prepared which has been previously transformed into the genetically engineered host E. coli DH10B (supplier: Source BioScience); 1.2) a recombinase expression vector was prepared, e.g. PKD46 (supplier: HonorGene, catalog number: HG-VJC0521, reference: Datsenko, KA, BL Wanner 2000. One-step inactivation of chromosomal genes in Escherichia coli K-12 using PCR products. Proc. Natl. Acad. Sci. U.S.A. 97 (12): 6640-5.), which comprises an arabinose inducible recombinase (e.g. derived from E. coli λ phage Red α/Red β/Red γ protein) expression element, replicon element of a temperature sensitive plasmid vector and an ampicillin resistance gene; 1.3) pKD46 was introduced into the host E. coli DH10B containing BAC1 using conventional electroporation and the E. coli was inoculated on LB solid media (supplier: Qingdao Haibo, Cat. No: HB0129) plates containing chloramphenicol and ampicillin overnight at 30° C.; 2) E. coli monoclones the following day was picked to the liquid LB medium (supplier: Qingdao Haibo, Cat. No.: HB0128) containing chloramphenicol (supplier: Sangon, Cat. No.: A100230) and ampicillin (supplier: Sangon, Cat. No.: A100339), shaking under the culturing condition of 30° C. for 16 hours, and the resulting strain was named E. coli A.

Step 2, 2.1) rpsL/tetA sequence as PCR template can be found in SEQ ID NO: 1. Design and synthesis of a forward primer and reverse primer as shown in FIG. 8A (supplier: Sangon), the forward primer comprising a 50 bp homology arm region HA1 to be knocked out at the 5 ′end and the primer region of rpsL/tetA at the 5′ end, the reverse primer comprising a 50 bp homology arm region HA2 to be knocked out at the 3 ′end and the primer region of rpsL/tetA at the 3′ end, the design principles of the primers being familiar to those skilled in the art; 2.2) DNA fragment with 50 bp homology arms and rpsL/tetA expression elements at both ends was obtained using polymerase chain reaction technique (PCR) using the forward and reverse primers described above (FIG. 8A); 2.3) E. coli A obtained in step 1.3) was re-inoculated in 3 ml of liquid LB medium containing chloramphenicol and ampicillin at a final concentration of OD600=0. 1, and 45 μl of 10% L (+) arabinose (supplier: Sangon, catalog number: A610071) was added for induction expression of recombinase, culturing in a shaker at 37° C. for 3-5 hours until the bacterial liquid OD600=0. 6; 2.4) the rpsL/tetA fragment obtained in step 2.2) was introduced into E. coli A in step 2.3) by means of electroporation transfection, the DNA fragment to be knocked out was replaced with rpsL/tetA by recombinase-mediated homologous recombination(FIG. 8B) in site-directed manner, followed by culturing overnight at 37° C. on LB solid medium plates containing chloramphenicol and tetracycline (supplier: Sangon, catalog number: A100422); 2.5) Colony clones obtained in step 2.4) were picked and cultured at 37° C. in LB liquid medium containing chloramphenicol and tetracycline, and E. coli clones containing BAC1 with the correct knock out of the region to be knocked out were identified by PCR identification and PCR product sequencing method using screening primers 1 and 2 in FIG. 8C, named E. coli B;

Step 3, 3.1) according to the step 1.3), pKD46 vector was introduced into the E. coli B clone obtained in step 2.5) containing BAC1 with the correct knock-out of the fragment to be knocked-out, named E. coli C; 3.2) a double stranded DNA fragment HA1-HA2 of 50 bp homology arm HA1 and 50 bp homology arm HA2 linked in sequence is designed and synthesized; 3.3) the DNA fragment of step 3.2) was introduced into E. coli C obtained in step 3.1) by means of electroporation, by reference to steps 2.3)-2.4), the rpsL/tetA fragment inserted when knocking out the region to be knocked out in BAC1 was replaced with the HA1-HA2 fragment in site-directed manner by recombinase-mediated homologous recombination (FIG. 8D), followed by culturing overnight at 37° C. on LB solid media plates containing chloramphenicol and streptomycin (supplier: Sangon, Cat. No. A100382); 3.4) Colony clones obtained in step 3.3) were picked and cultured at 37° C. in LB liquid medium containing chloramphenicol and streptomycin, and E. coli clones containing BAC1 with the correct knockout of rpsL/tetA region were identified by PCR identification and PCR product sequencing method using screening primers 1 and 2 in FIG. 8E;

Step 4, if BAC1 contains multiple pseudogenes or open reading frame regions to be knocked out, repeating Steps 1 to 3 can separately knock out each region to be knocked out until all pseudogenes or open reading frame regions of interest are knocked out in BAC1. When segmented human immunoglobulin variable region DNA is inserted into different BAC vectors, respectively, knockout steps of pseudogenes and open reading frame regions can be independently implemented in different BAC vectors, respectively, and finally retained DNA fragments can be spliced together by the steps in the publication “Assisted large fragment insertion by Red/ET-recombination (ALFIRE)—an alternative and enhanced method for large fragment recombineering”.

In particular embodiments of the present invention, generally, for “partial or entire knockout” or “partial or entire deletion”, 10-100% (preferably 15-95%, 20-90%, such as 25%, 30%, 35%, 40%, 45%, 50%, 55%, 60%, 65%, 70%, 72%, 75%, 78%, 80%, 83%, 85% or 88%) of the pseudogenes and/or open reading frame genes are knocked out or deleted, the percentage based on the total number of pseudogenes and open reading frame genes of the human immunoglobulin variable region gene.

For example, in particular embodiments of the present invention, at least 80%, 81%, 82%, 83%, 84%, 85%, 86%, 87%, 88%, 89%, 90%, 91%, 92%, 93%, 94%, 95%, 96%, 97%, 98%, 99% or 100% of the pseudogenes and/or open reading frames are knocked out.

For example, in one embodiment, 10-25 pseudo-V-genes and/or open reading frame genes are knocked out or deleted; in another embodiment, 10, 15 or 25 pseudo-V-genes and/or open reading frame genes are knocked out or deleted.

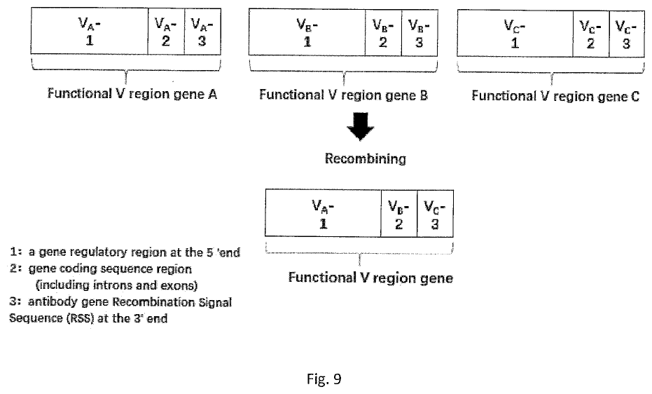

During knocking out a pseudogene and/or open reading frame in a region of a human immunoglobulin gene cluster, the regulatory region, gene coding region and antibody gene recombination signal sequence of the retained functional gene fragment may be contiguous segments derived from the same immunoglobulin variable region gene, it is also possible to recombine the regulatory region, gene coding region and antibody gene recombination signal sequence derived from different immunoglobulin genes, for example the regulatory region is derived from the 5 ′end regulatory region of the human VA gene, the coding sequence is derived from the VB gene, and the antibody gene recombination signal sequence is derived from the VC gene (as shown in FIG. 9).

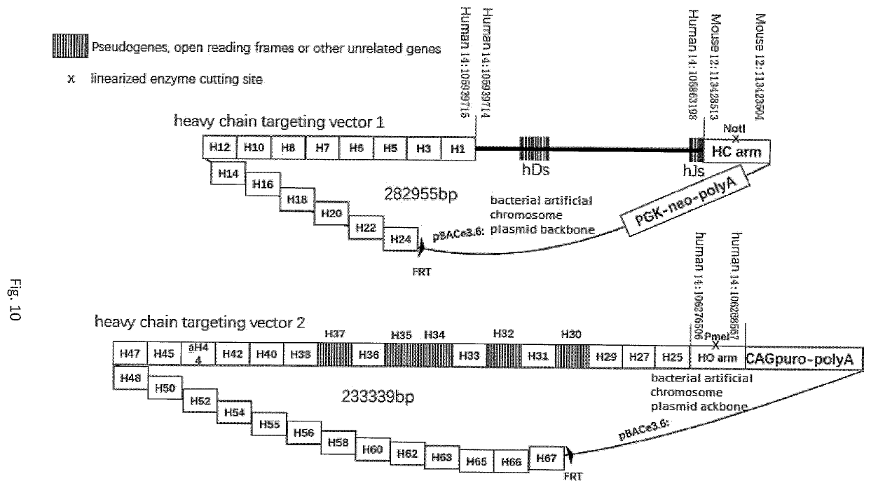

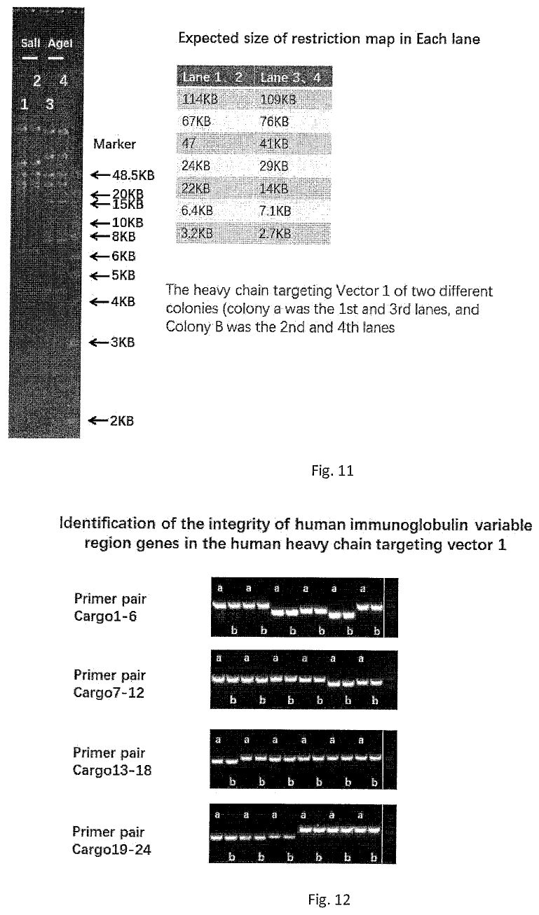

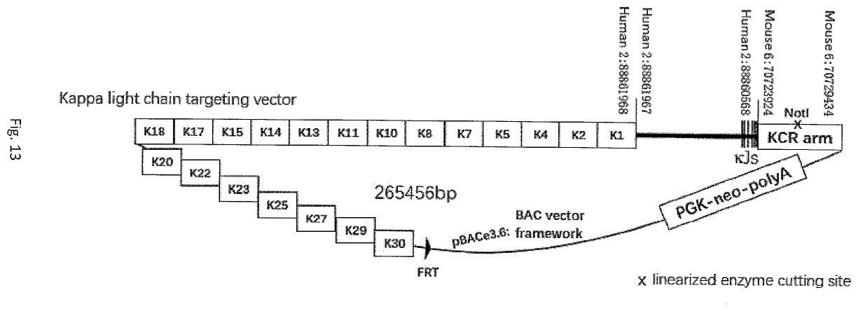

In one of the embodiments, the heavy chain targeting vector 1 as shown in FIG. 10 was constructed, DNA fragments of 10 of H2, H4, H9, H11, H13, H15, H19, h21, H23, H17 comprising pseudo-V_{H}-region gene or open reading frame V_{H} region gene in the human immunoglobulin heavy chain V region in Table 1 were knocked out according to the steps described above, and H1, H3, H5, H6, H7, H8, H10, H12, H14, H16, H18, H20, H22, H24 comprising functional human heavy chain V_{H} region genes of IGHV6-1, IGHV1-2, IGHV1-3, IGHV4-4, IGHV7-4-1, IGHV2-5, IGHV3-7, IGHV1-8, IGHV3-9, IGHV3-11, IGHV3-13, IGHV3-15, IGHV1-18, IGHV3-20, IGHV3-21, IGHV3-23, and sequences including human immunoglobulin heavy chain D_{H} and J_{H} gene regions derived from between 105863198 and 105939714 of chromosome 14, and sequence derived between 113428513 to 113423504 of murine chromosome 12 with insertion of unique linearized enzyme cutting site Notl at 113426008 site, were retained and ligated in sequence as shown in FIG. 10. The heavy chain targeting vector 1 carries the neomycin resistance expression element PGK-neo-polyA, the recombinase recognition site FRT and the pBACe3.6 bacterial artificial chromosome plasmid backbone. Two different ways were taken to identify if there's deletion of the DNA fragments after the vector was constructed. The first way of identification is to judge if the cut profile is as expected by pulse electrophoresis after digesting with Sail or Agel, as shown in FIG. 11, each fragment size after digestion by two different sets of enzymes on heavy chain targeting vector 1 derived from two different colony clones a and b is in line with expectations, indicating that the two clonally derived heavy chain targeting vector 1 have a lower risk of large fragments of DNA deletion; a second way of identification is as shown in FIG. 12, 24 pairs of primers Cargo1-Cargo24 were randomly designed in the heavy chain targeting vector 1 (as shown in Table 2), the distance of each pair of primers in the vector is approximately 10 KB, and then PCR amplification of these 24 pairs of primers is performed with the heavy chain targeting vector 1 derived from two different colony clone a and b, which can be illustrated by the size of the product and whether the product is present, that the two clonally derived heavy chain targeting vector 1 have a lower risk of large fragments of DNA deletion.

TABLE 2
human immunoglobulin gene fragment specific primer pair
Cargo 1-24 (column of SEQ ID NO: in the table, even
numbers correspond to forward primer, odd numbers
correspond to reverse primer)

| Primer | | | SEQ ID | Product |
|---|---|---|---|---|
| name | Forward primer | Reverse primer | NO | size |
| Cargo1 | gcaggagagaggttgtgagg | gtgacccattcgagtgtcct | 2, 3 | 505 bp |
| Cargo2 | actggtccctggtgccttat | ccttgagcaagacccagtgt | 4, 5 | 497 bp |
| Cargo3 | gaactggggcatctctcgga | tgactggactcgcagggttt | 6, 7 | 263 bp |
| Cargo4 | gtcccttttgctggctttggtc | ggtggccccataacacaccta | 8, 9 | 333 bp |
| Cargo5 | tccagaagtggaagcgttta | aaaccccctggaaatcatagta | 10, 11 | 197 bp |
| Cargo6 | ctctctctggttcccagcac | ggcaggctgactttcactct | 12, 13 | 499 bp |
| Cargo7 | ctgagggccgatggtactaa | acactctggggccatgtaag | 14, 15 | 505 bp |
| Cargo8 | ctaggccctggtaaccaaca | agttctgaatggggctgaga | 16, 17 | 503 bp |
| Cargo9 | aggcatctcggcaaaaatta | ggcatggaggaaatgacaaa | 18, 19 | 519 bp |
| Cargo10 | gggcatggacatagcagatt | gcgcaatgaactggtacaaa | 20, 21 | 503 bp |
| Cargo11 | gcccactccacaattcctaa | ctgtgactttccccacaggt | 22, 23 | 358 bp |
| Cargo12 | ggtgttgcatctgtggtgag | ggcttctctggaaatgcaag | 24, 25 | 400 bp |
| Cargo13 | agcgaaaggagtcattcaaa | ggttggtttccaggttgtgt | 26, 27 | 392 bp |
| Cargo14 | ttttgctccttcctgtgtcc | atccagcaccacagtcacaa | 28, 29 | 501 bp |
| Cargo15 | aacaaaagcaggcgttcact | cacccatccactgcctattt | 30, 31 | 491 bp |
| Cargo16 | ctcagtaagggagcgcatct | gggctgagaaaagggaagtc | 32, 33 | 500 bp |
| Cargo17 | atggggcacaaaggtatgtt | ccagtgtggtctcgatttcc | 34, 35 | 514 bp |
| Cargo18 | agggtcccagataggttgct | cctgaaagatcgggctgtaa | 36, 37 | 520 bp |
| Cargo19 | gctccctaccatccattcaa | gttcaaacaaaaggcccaga | 38, 39 | 303 bp |
| Cargo20 | ttactttgcaggggaaccac | tgagtgttcctgaccctcct | 40, 41 | 300 bp |
| Cargo21 | gcaaatgctgtttatggatca | gcaaatggcagcatctttct | 42, 43 | 347 bp |
| Cargo22 | ctctcacccagggaaaacag | gataaccagacatgttgggtca | 44, 45 | 495 bp |
| Cargo23 | ccttgctaggttggggaagt | ccagcaacagaacaaagctg | 46, 47 | 501 bp |
| Cargo24 | ggtgagaggcctttggagat | catcacaccatgttcccatt | 48, 49 | 498 bp |

In another embodiment, the heavy chain targeting vector 2 as illustrated in FIG. 10 was constructed, characterized in that, in the sequence between 106879844 and 106268567 of human chromosome number 14, DNA fragments of 15 of sequence number H26, H28, H35, H39, H41, H43, H46, H49, H51, H53, H57, H59, H61, H64, H68 comprising the pseudo-V_{H}-gene or the open reading frame V_{H} region gene as described in Table 1 were knocked out in E. coli by the above-said procedure, fragments of 25 of sequence number H25, H27, H29, H31, H33, H36, H38, H40, H42, H44, H45, H47, H48, H50, H52, H54, H55, H56, H58, H60, H62, H63, H65, H66, H67 comprising the functional V_{H} region gene as described in Table 1, as well as pseudo-V_{H}-region gene fragments of H30, H32, H34, H37 and the H35 gene fragment as described in Table 1, and the sequence between 106276506 to 106268567 of human chromosome 14 as homology arms with insertion of the unique linearized enzyme cutting site Pmel at site 106273423 of the homology arm were retained, and these DNA fragments were sequentially ligated as shown in FIG. 10; the heavy chain targeting vector 2 carries the puromycin resistance expression element CAGpuro-polyA, the recombinase recognition site FRT and the pBACe3.6 bacterial artificial chromosome plasmid backbone. The Heavy Chain Targeting Vector 2 was used to introduce more V region gene fragments on the Heavy Chain Targeting Vector 2 into Chromosome 12 of Mouse Embryonic Stem Cell with a sequence that was carried into between 106276506 and 106268567 of human chromosome 14 by the Heavy Chain Targeting Vector 1 as homology arm, after the directed introduction of the Heavy Chain Targeting Vector 1 into position between 113428513 and 113423504 of mouse chromosome 12.

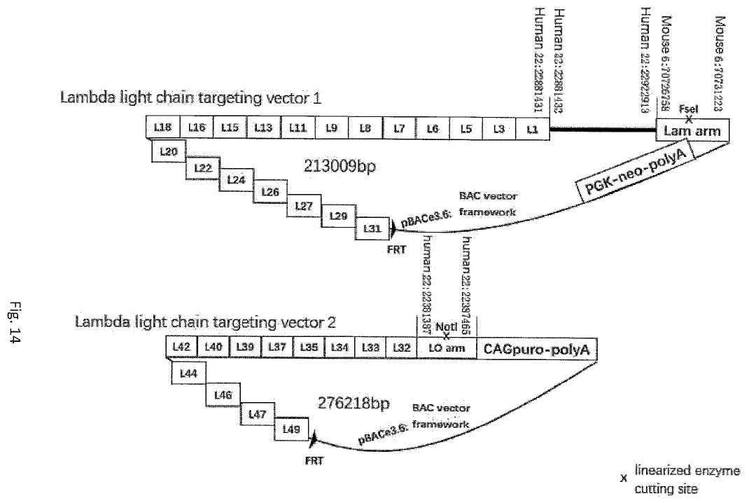

In another embodiment, a Kappa light chain targeting vector as depicted in FIG. 13 was constructed, characterized in that, in the sequence between 89333431 and 88860568 of human chromosome 2, 10 regions of sequence number K3, K6, K9, k12, K16, K19, K21, K24, K26, K28 comprising pseudogenes or open reading frames as described in Table 1 were knocked out in E. coli according to the above-said procedure, 20 of the sequence number K1, K2, K4, K5, K7, K8, K10, K11, K13, K14, K15, K17, K18, K20, K22, K23, K25, K27, K29, K30 comprising functional human Kappa light chain V_{L} region gene as described in Table 1 and human immunoglobulin Kappa light chain J_{L} gene region between 88861967 and 88860568 of human chromosome 2 were retained, together with the sequence between 70723924 and 70729434 of mouse chromosome 6 as homology arms with insertion of the unique linearized enzyme cutting site Notl at site 70726623 of the homology arm, wherein the Kappa light chain targeting vector carries the neomycin resistance expression element PGK-neo-polyA, the recombinase recognition site FRT and the pBACe3.6 bacterial artificial chromosome plasmid backbone.

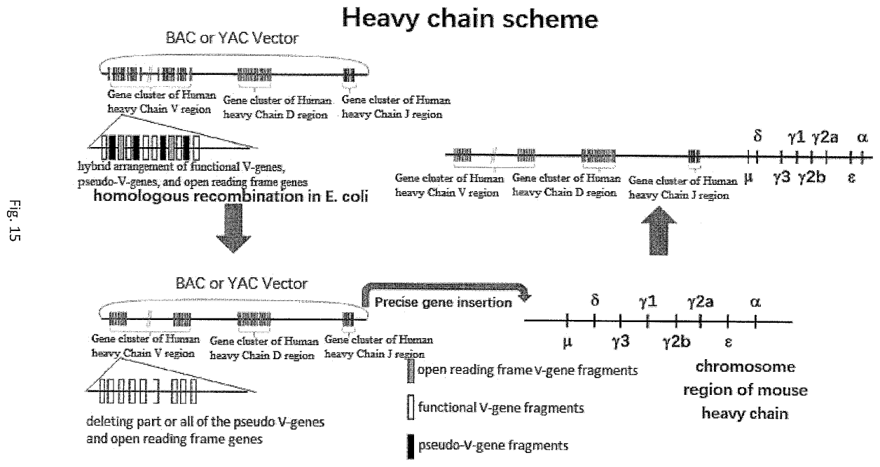

In another embodiment, lambda light chain targeting vectors 1 and 2 were constructed as shown in FIG. 14, lambda light chain targeting vector 1 comprises a sequence derived from between 22881432 and 22922913 in human chromosome 22 that contains human immunoglobulin Lambda light chain J-C gene region, 12 regions of sequence number L2, L4, L10, L12, L14, L17, L19, L21, L23, L25, L28, L30 comprising pseudogenes or open reading frames as described in Table 1 were knocked out in E. coli according to the above-said procedure, 19 regions of the sequence number L1, L3, L5, L6, L7, L8, L9, L11, L13, L15, L16, L18, L20, L22, L24, L26, L27, L29, L31 comprising the functional human Lambda light chain V_{L} region gene as described in Table 1, and sequence between 70726758 and 70731223 of mouse chromosome 6 as homology arms with insertion of the unique linearized enzyme cutting site Fsel at site 70729051 of the homology arm, were retained, wherein the Lambda light chain targeting vector 1 carries the neomycin resistance expression element PGK-neo-polyA, recombinase recognition site FRT, and the pBACe3.6 bacterial artificial chromosome plasmid backbone; for lambda Light Chain Targeting Vector 2, 7 regions of sequence number L36, L38, L41, L43, L45, L48, L50 comprising pseudogenes or open reading frames as described in Table 1 were knocked out in E. coli according to the above-described steps, 12 regions of the sequence number L32, L33, L34, L35, L37, L39, L40, L42, L44, L46, L47, L49 comprising the functional human Lambda light chain V_{L} region gene as described in Table 1, and sequence between 22381387 and 22387465 of human chromosome 22 as homology arm were retained, wherein the homology arm was inserted into a linearized enzyme cutting site Notl at site 22383529, the Lambda light chain targeting vector 2 carrying puromycin resistance expression element CAGpuro-polyA, recombinase recognition site FRT, and pBACe3.6 bacterial artificial chromosome plasmid backbone. The use thereof is to introduce more V region gene fragments on the Lambda light chain targeting vector 2 into chromosome 6 of mouse embryonic stem cell with a sequence that was carried into between 22381387 and 22387465 of human chromosome 22 by the Lambda light chain targeting vector 1 as homology arm, after directed introduction of Lambda light chain targeting vector 1 into position 70726758 and 70731223 of mouse chromosome 6.

The DNA integrity identification method for the heavy chain targeting vector 2, the kappa light chain targeting vector, and the lambda light chain targeting vectors 1 and 2 applied pulse electrophoresis of vector restriction enzyme digestion fragment, and the PCR identification method of random small fragment is similar to that of the heavy chain targeting vector 1.

### Example 5: Directed Introduction of Targeting Vectors into Mouse Embryonic Stem Cells by Homologous Recombination

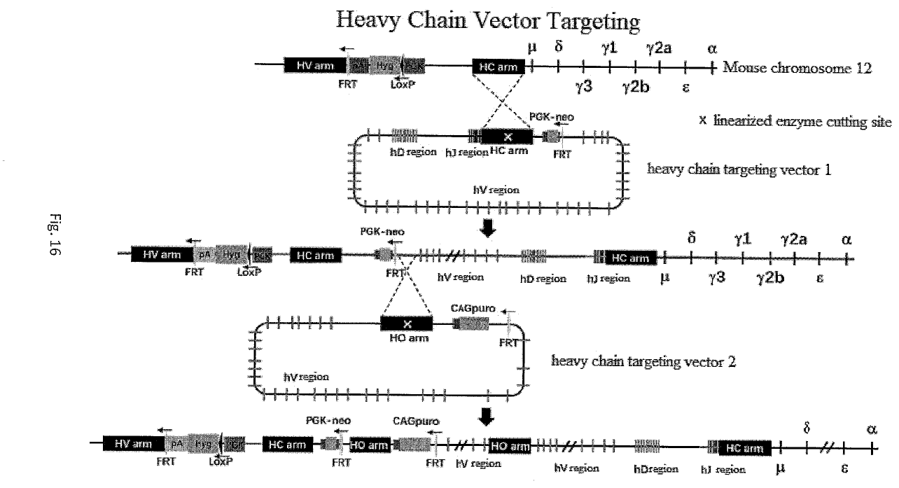

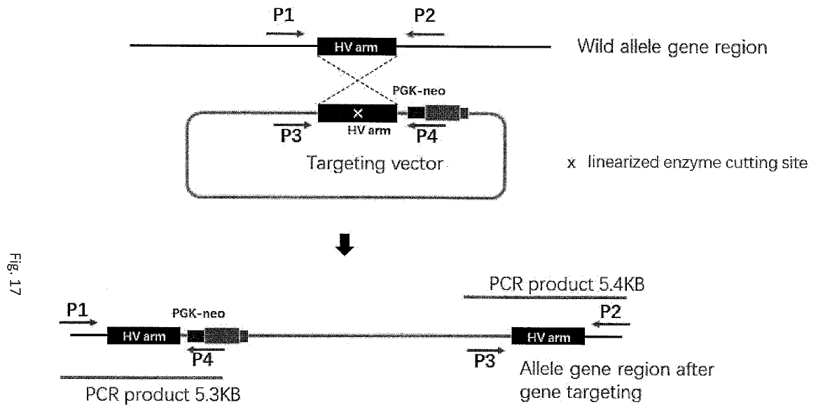

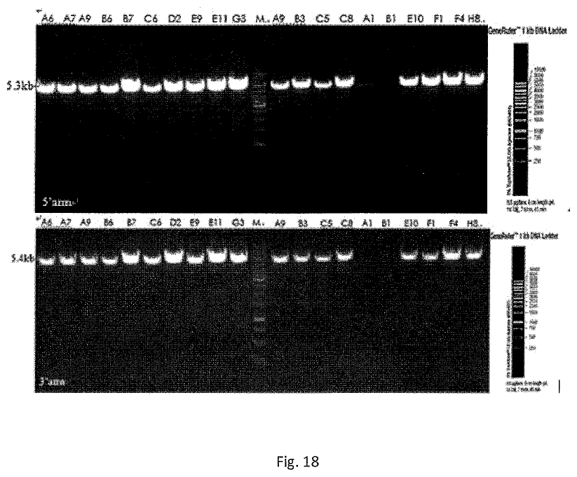

In one example, the target cell is a mouse embryonic stem cell of Example one with its chromosome region between 113428530 and 116027502 removed through a two-step gene targeting strategy using recombinase Cre, and the targeting vector used is the heavy chain targeting vector 1 (as shown in FIG. 10), and the directed introduction process is described with reference to FIG. 15 and FIG. 16, with the following steps:
Step 1, 1-2×10⁷ target mouse embryonic cells were transfected with 50 μg of linearized heavy chain targeting vector 1 by electroporation (240 V, 250 uF, Bio-rad Gene Pulser), wherein the method of electroporation transfection is familiar to those skilled in the art. Successfully transfected cell clones were selected with 225 μg/ml neomycin for 7 days within 24-48 hours post the transfection depending on the resistance gene carried on the targeting vector. Antibiotic resistant cell clones were picked into 96-well culture plates for scale-up and genotype identification.
Step 2, conventional PCR amplification was carried out by designing forward and reverse primers outside the homology arm regions as shown in FIG. 17 after extracting genomic DNA from part of the antibiotic-resistant cells obtained in step 1, 1) primers P1 and P2 were designed at the 5 ′and 3′ ends of the target homology arm of the wild-type allele, respectively, and the sequences of both primers are outside the homology arm region; 2) Primers P3 and P4 were designed at the 5 ′and 3′ ends of the homology arm of the targeting vector, respectively, wherein the sequences of both primers are outside the homology arm region; 3) when the linearized targeting vector was inserted into the target homology arm site of the wild-type allele by homologous recombination, DNA fragments P1P4 and P3P2 with fragment sizes larger than the size of the homology arm segment could be amplified by PCR using P1 and P4 primer pair, and P3 and P2 primer pair, respectively, while DNA fragments with fragment sizes larger than the size of the homology arm segment in a PCR system with only wild-type allele template or targeting vector template, cannot be amplified using the P1 and P4 primer pair, and P3 and P2 primer pair, respectively. Whether the targeting vector in the protocol was accurately inserted into the target homology arm region of the wild-type allele by homologous recombination was judged by analyzing the fragment size and sequence of the two PCR products, P1P4 and P3P2. As shown in FIG. 18, the size of the P1P4 product was 5.3 KB and the size of the P3P2 product was 5.4 KB after directed introduction of the heavy chain targeting vector 1 into the target mouse embryonic stem cells, which showed that multiple cell clones with successful directed introduction into the target region of the mouse embryonic stem cells by the directed introduction. The amplification products P1P4 and P3P2 can be further confirmed by DNA sequencing.

The integrity of human immunoglobulin variable region genes in the human heavy chain targeting vector 1 introduced into mouse embryonic stem cells can be identified by PCR with as template the host cell genome, Cargo 1-24 as shown in Table 2 as primers were used for PCR amplification and the PCR products of the expected size were obtained using Cargo 1-24, indicating that mouse embryonic stem cell clones were those successfully introducing human immunoglobulin variable region loci without risk of significant DNA fragment deletion.

In one of the embodiments, as shown in FIGS. 15 and 16, 16 human V_{H} functional genes, 27 human D_{H} genes and 6 human J_{H} genes of human immunoglobulin heavy chain variable region were introduced via heavy chain targeting vector 1 into the mouse embryonic stem cell after all steps shown in Example 1 were completed, and the embryonic stem cell clone was further introduced with an additional 25 human V_{H} functional genes via the heavy chain targeting vector 2.

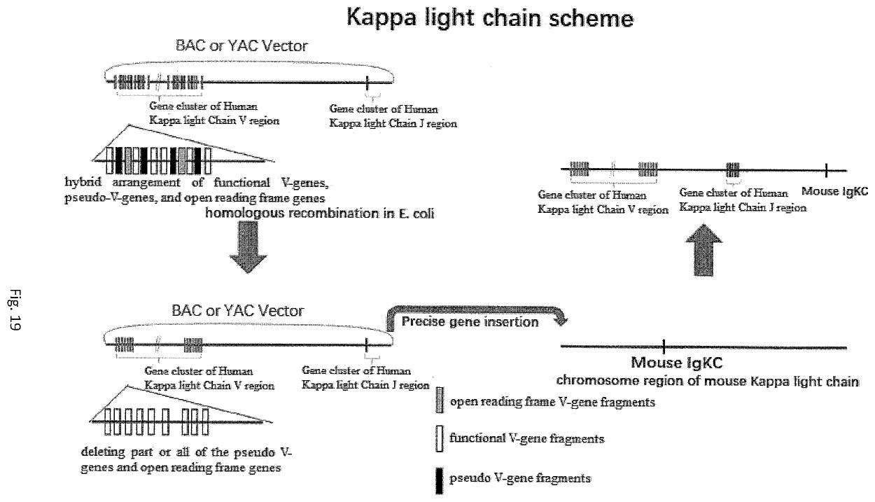

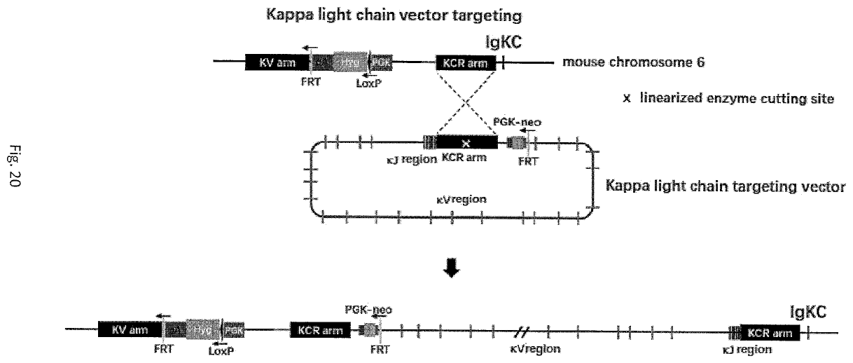

In one of the examples, as shown in FIGS. 19 and 20, 20 human kappa V_{L} functional genes and 5 kappa J_{L} genes of the human immunoglobulin kappa light chain variable region were introduced via the kappa light chain targeting vector into the mouse embryonic stem cell after all the steps shown in Example 2 were completed.

In one of the embodiments, as shown in FIGS. 21 and 22, 19 human lambda V_{L} functional gene, all 7 lambda J_{L} genes and lambda C L genes of the human immunoglobulin lambda light chain variable region were introduced via lambda light chain targeting vector 1 into the mouse embryonic stem cell after all the steps shown in Example 3 were completed, and an additional 12 human lambda V_{L} functional genes were further introduced into the embryonic stem cell clone via lambda targeting vector 2. FLP expression was introduced into the cell after introduction of the lambda light chain targeting vector 1 was completed, allowing recombination between the two FRT sites to knock out the sequence between the two FRTs such that residual non-human source DNA sequences derived from the Lambda light chain targeting vector and the coding sequence for mouse endogenous kappa CL were removed.

Before introducing the gene comprising human immunoglobulin variable region into the mouse embryonic stem cells in the embodiments as shown in FIGS. 15-16, 19-20, 21-22, part or all of the pseudo-V-region genes and/or open reading frame V-region genes in the human immunoglobulin variable region V-region genes were knocked out, the fragment size of the V region gene ultimately introduced into mouse embryonic stem cells thus accounts for only a fraction of the size of the human immunoglobulin V region in the human genome database of the version GRCh38.p13 from ENSEMBL, the percentage of which is summarized in Table 3 below:

TABLE 3
Percentage of V-region gene fragments introduced into cells after completion of
each targeting step in Example 5 over total length of corresponding V-region

| | Length of V | Total Length of | Percentage of V |
|---|---|---|---|
| | region gene | Corresponding V | region genes |
| Completion Step | introduced | Region Gene Regions | introduced |
| after completion of introduction of | 151362 bp | 940130 bp | 16.10%⁽¹⁾ |
| heavy chain targeting vector 1 | | | |
| after completion of introduction of | 357803 bp | 940130 bp | 38.06%⁽¹⁾ |
| heavy chain targeting vector 1 and | | | |
| heavy chain targeting vector 2 | | | |
| after completion of introduction of | 250235 bp | 471464 bp | 53.08%⁽²⁾ |
| kappa light chain targeting vector | | | |
| after completion of introduction of | 158751 bp | 858318 bp | 18.50%⁽³⁾ |
| lambda light chain targeting vector 1 | | | |
| after completion of introduction of | 272518 bp | 858318 bp | 31.75%⁽³⁾ |
| lambda light chain targeting vector | | | |
| 1 and lambda light chain targeting | | | |
| vector 2 | | | |

Note:
⁽¹⁾the complete size of the human heavy chain V region was calculated to be 940130 bp, according to positions between 105939715 and 106879844 of chromosome 14 of ENSEMBL GRCh38.p13;
⁽²⁾the complete size of human kappa light chain proximal V regions was calculated to be 471464 bp, according to positions between 88861968 and 89333431 of chromosome 2 of ENSEMBL GRCh38.p13;
⁽³⁾the complete size of the human lambda light chain V region was calculated to be 858318 bp, according to positions between 22023114 and 22881431 of chromosome 22 of ENSEMBL GRCh38.p13.

### Example 6: Conversion and Propagation of Mouse Embryonic Stem Cells

Techniques for conversion of gene-edited mouse embryonic stem cells into transgenic mice are familiar to those skilled in the art. When genotypically qualified mouse embryonic stem cell clones have no increase or decrease in chromosome number as determined by karyotype detection, mouse embryonic stem cell of this clone was treated and diluted to injection density and injected into blastocoel cavities of approximately 50 blastocysts at 2.5-3.5 day old, the micro-injected blastocysts were then returned to the oviduct or uterus of surrogate mice, after the mice were born, the genotype of the progeny chimeras was identified by PCR using gene editing specific primers with the tail DNA as template to determine whether the gene edited mouse embryonic stem cells contribute to the somatic cells of the chimeric progeny mice. Progeny chimeric mice and wild type mice of different sex bred in the same cage to obtain filial generation mice (F1), the same PCR method was used to identify the genotype of F1 generation mice, whether the genetically edited mouse embryonic stem cells were capable of germline transmission can be determined, and primer design principles and protocol for genotype identification of F0 and F1 generation mouse are familiar to those skilled in the art.

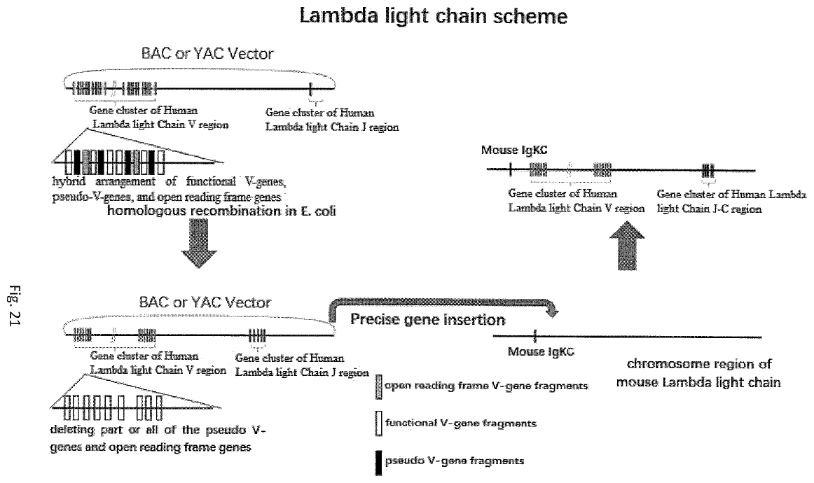

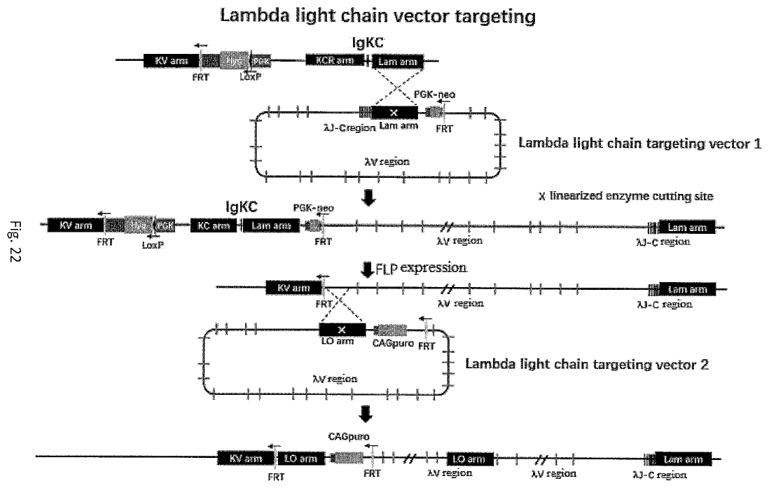

In one of the embodiments, mouse embryonic stem cells obtained in Example 5, to which a human immunoglobulin heavy chain variable region locus were successfully introduced and identified by PCR amplification of Cargo 1-24 as shown in Table 2 and karyotype detected, were injected into the blastocoel of a 2.5-3.5 day-old mouse embryo following the methods described above and F1 mice were finally obtained; in another embodiment, introduction of the Kappa light chain-targeting vector shown in FIG. 13 into mouse embryonic stem cells according to the scheme shown in FIGS. 19 and 20 also obtained F1 mice, both the F1 mice bred by crossing to become mice homozygous for both the site of the heavy chain-targeting vector 1 and the site of the Kappa light chain-targeting vector; in another embodiment, the introduction of Lambda light chain targeting vectors 1 and 2 as shown in FIG. 14 into mouse embryonic stem cells according to the schemes shown in FIGS. 21 and 22 also obtained F1 mice. The features of several transgenic mice obtained according to the present invention are shown in Table 4.

TABLE 4
Characteristics of transgenic mice obtained according to the present invention

| Mouse | |
|---|---|
| species | Features |
| Transgenic | Obtained by breeding mice after directed introducing of the heavy chain |
| mice | targeting vector 1 and the Kappa light chain targeting vector respectively, and |
| Group 1 | both the sites of the two vectors are homozygous, the mice comprising 16 |
| | human immunoglobulin heavy chain variable region V_{H} genes: IGHV6-1, |
| | IGHV1-2, IGHV1-3, IGHV4-4, IGHV7-4-1, IGHV2-5, IGHV3-7, IGHV1-8, IGHV3-9, |
| | IGHV3-11, IGHV3-13, IGHV3-15, IGHV1-18, IGHV3-20, IGHV3-21, IGHV3-23 and |
| | 20 human immunoglobulin Kappa light chain variable region V_{L} genes: IGKV4-1, |
| | IGKV5-2, IGKV1-5, IGKV1-6, IGKV1-8, IGKV1-9, IGKV3-11, IGKV1-12, IGKV3-15, |
| | IGKV1-16, IGKV1-17, IGKV3-20, IGKV6-21, IGKV2-24, IGKV1-27, IGKV2-28, |
| | IGKV2-30, IGKV1-33, IGKV1-39, IGKV2-40. |
| Transgenic | Obtained by breeding mice after directed introducing of heavy chain targeting |
| mice | vectors 1 and 2 and Kappa light chain targeting vector respectively, and the sites |
| Group 2 | of the vectors are all homozygotes, the mice comprising 41 human |
| | immunoglobulin heavy chain variable region V_{H} genes: IGHV6-1, IGHV1-2, |
| | IGHV1-3, IGHV4-4, IGHV7-4-1, IGHV2-5, IGHV3-7, IGHV1-8, IGHV3-9, IGHV3-11, |
| | IGHV3-13, IGHV3-15, IGHV1-18, IGHV3-20, IGHV3-21, IGHV3-23, IGHV1-24, |
| | IGHV2-26, IGHV4-28, IGHV3-30, IGHV4-31, IGHV3-33, IGHV4-34, IGHV4-39, |
| | IGHV3-43, IGHV1-45, IGHV1-46, IGHV3-48, IGHV3-49, IGHV5-51, IGHV3-53, |
| | IGHV1-58, IGHV4-59, IGHV4-61, IGHV3-64, IGHV3-66, IGHV1-69, IGHV2-70, |
| | IGHV3-72, IGHV3-73, IGHV3-74 and 20 human immunoglobulin Kappa light |
| | chain variable region V_{L} genes: IGKV4-1, IGKV5-2, IGKV1-5, IGKV1-6, IGKV1-8, |
| | IGKV1-9, IGKV3-11, IGKV1-12, IGKV3-15, IGKV1-16, IGKV1-17, IGKV3-20, |
| | IGKV6-21, IGKV2-24, IGKV1-27, IGKV2-28, IGKV2-30, IGKV1-33, IGKV1-39, |
| | IGKV2-40. |
| Transgenic | Obtained by breeding mice after directed introducing of heavy chain targeting |
| mice | vectors 1 and 2 and Lambda light chain targeting vectors 1 and 2, respectively, |
| Group 3 | and the sites of the vectors are all homozygotes, the mice comprising 41 human |
| | immunoglobulin heavy chain variable region V_{H} genes: IGHV6-1, IGHV1-2, |
| | IGHV1-3, IGHV4-4, IGHV7-4-1, IGHV2-5, IGHV3-7, IGHV1-8, IGHV3-9, IGHV3-11, |
| | IGHV3-13, IGHV3-15, IGHV1-18, IGHV3-20, IGHV3-21, IGHV3-23, IGHV1-24, |
| | IGHV2-26, IGHV4-28, IGHV3-30, IGHV4-31, IGHV3-33, IGHV4-34, IGHV4-39, |
| | IGHV3-43, IGHV1-45, IGHV1-46, IGHV3-48, IGHV3-49, IGHV5-51, IGHV3-53, |
| | IGHV1-58, IGHV4-59, IGHV4-61, IGHV3-64, IGHV3-66, IGHV1-69, IGHV2-70, |
| | IGHV3-72, IGHV3-73, IGHV3-74 and 31 human immunoglobulin Lambda light |
| | chain variable region V_{L} genes: IGLV3-1, IGLV4-3, IGLV2-8, IGLV3-9, IGLV3-10, |
| | IGLV2-11, IGLV3-12, IGLV2-14, IGLV3-16, IGLV2-18, IGLV3-19, IGLV3-21, |
| | IGLV2-23, IGLV3-25, IGLV3-27, IGLV1-36, IGLV5-37, IGLV1-40, IGLV7-43, |
| | IGLV1-44, IGLV5-45, IGLV7-46, IGLV1-47, IGLV9-49, IGLV1-51, IGLV5-52, |
| | IGLV10-54, IGLV6-57, IGLV4-60, IGLV8-61, IGLV4-69. |

The sequences of the variable region V genes in Table 4 refer to Table 1.

### Example 7: Studies of B Cell Development in Transgenic Mice

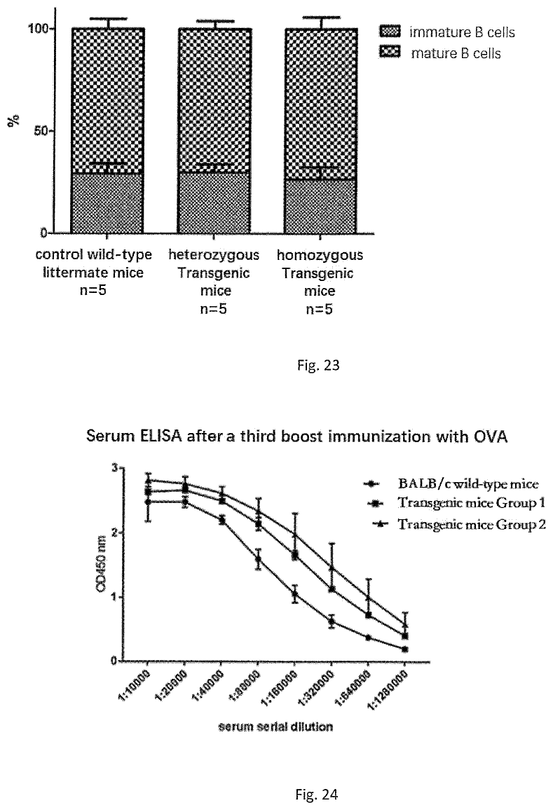

In most mammals, the spleen is a B cell-abundant organ, and analysis of B cells in the spleen can determine whether B cell development is normal in that animal. Techniques for obtaining (transgenic mouse obtained in Example 6) mouse spleens and isolating spleen cells, and flow cytometric analysis of spleen-derived cells are familiar to those skilled in the art. The spleens of transgenic mice group 1 in Example 6 (homozygous transgenic mice group in FIG. 23) were collected without immunization and spleen cells were isolated, and flow cytometric analysis thereof compared to that of spleen cells derived from heterozygous transgenic mice and wild-type littermate control mice, selecting B220 ^{positive}/IgM^{high}/IgD^{low} cells as immature B cells, B220^{positive}/IgM^{low to medium}/IgD^{high} as mature B cells, as shown in FIG. 23, the wild-type littermate control mice, heterozygous transgenic mice and homozygous transgenic mice group are not statistically different in the ratio of mature and immature B cells, illustrating that B cell development of transgenic mice group 1 obtained according to the present invention is normal. Similarly, transgenic mice groups 2 and 3 are not statistically different in the ratio of mature and immature B cells from either wild-type littermate control mice or heterozygous transgenic mice, indicating that B cells develop normally.

### Example 8: Studies of Immune Response of Transgenic Mice

One of the aims of the present invention is to obtain transgenic animals expressing antibodies whose variable region encoding genes are human-derived, and such transgenic animals can normally produce an antigen-specific immune response upon antigenic stimulation. Groups 1 and 2 of transgenic mice obtained in Example 6 and control group of wild-type BALB/c mice were immunized with ovalbumin (OVA, supplier: Sigma-Aldrich, catalog number: A5503) at the same dose with the same immunization protocol. After the third boost of immunization, mouse sera were collected for an OVA-specific enzyme-linked immunosorbent assay (ELISA), wherein antigen-specific enzyme-linked immunosorbent assay methods are familiar to those skilled in the art. As a result, shown in FIG. 24, both groups 1 and 2 of transgenic mice were able to generate antigen-specific immune responses superior to wild-type BALB/c mice.

### Example 9. Application Example of Transgenic Mice in Fully Human Antibody Candidate Drug Discovery

As antibodies with variable region encoding genes derived from human immunoglobulin variable region rearrangements can be generated, one of the important uses of the mouse model of the present invention is discovery of full human antibody drug candidates. Techniques for staged stimulation of transgenic mice with a particular antigen to develop a specific immune response against the particular antigen, and cell fusion of the mouse spleen and/or lymph nodes to obtain immortalized and sustainable antibody-secreting hybridomas are familiar to those skilled in the art.

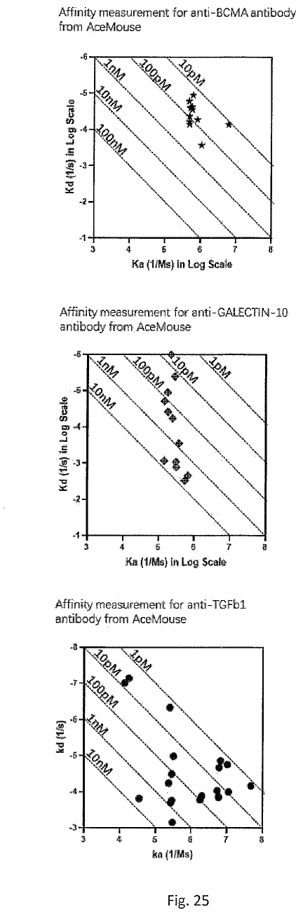

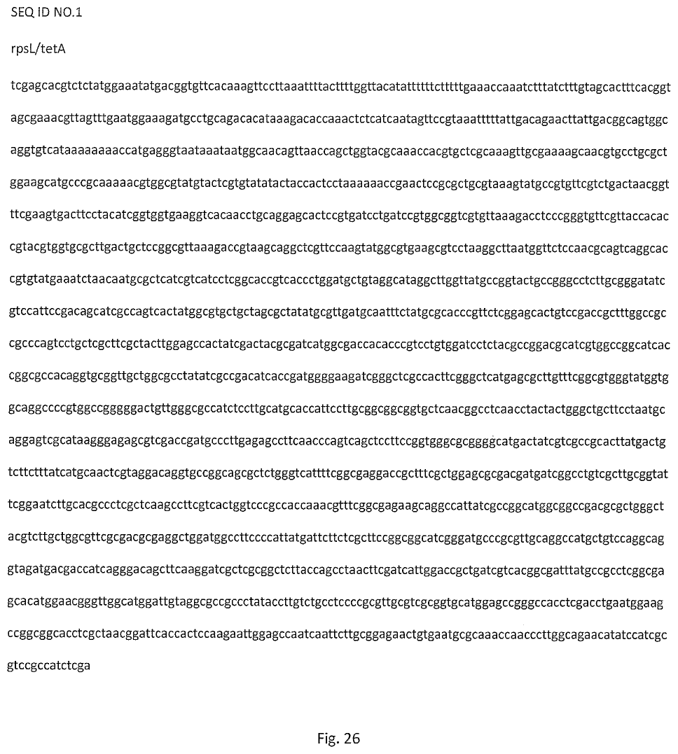

Gene editing for immunoglobulin-encoding gene clusters has an important negative impact on B cell development in mice, wherein one of the possible phenomena objectively reflected is that a specific immune response of mice against an antigen can only produce antibodies with low affinity. Using mouse models of groups 1, 2 and 3 of transgenic mice obtained in Example 6 according to the present invention, 12, 12 and 20 different antigen-specific monoclonal antibodies were obtained using hybridoma technology against three different targets, human BCMA, GALECTIN-10 or TGFb1, respectively. Using BIAcore T200 (GE Healthcare) to determine the affinity levels of these antibodies (FIG. 25), it can be concluded that these antibodies obtained from the mouse models of the present invention were able to reach high affinity levels in the pM range for all three different targets, with measured affinity levels in the range of 10 pM-1 nM, 1 pM-10 nM, 1 pM-10 nM, respectively.

Transgenic mice prepared using the methods of the present invention show marked potential, whether at the level of development of B cells, or at specific immune responses to antigens as well as at the level of affinity of antibodies, the effects are unexpected to those skilled in the art.

The particular embodiments disclosed in the foregoing should not limit the scope of the present invention and claims, as these embodiments are intended to exemplify several aspects of the present invention. Any equivalent embodiments are intended to fall within the scope of the present invention. Numerous other modifications to the present invention, in addition to those already described herein, will be apparent to those skilled in the art from the foregoing description and are intended to fall within the scope of the invention.

1. A genetically engineered recombinant genome of non-human mammalian cell, endogenous immunoglobulin variable region genes in the genome are partially or entirely replaced by human immunoglobulin variable region genes, wherein part or all of the pseudogenes and/or open reading frames of the human immunoglobulin variable region genes are knocked out.

2. The genetically engineered recombinant genome of non-human mammalian cell according to claim 1, wherein the human immunoglobulin variable region genes include coding and non-coding sequences for human heavy chain functional V_{H}, D_{H}, J_{H}, and/or coding and non-coding sequences for human light chain functional V_{L}, J_{L}, the light chain is a kappa or lambda light chain.

3. The genetically engineered recombinant genome of non-human mammalian cell according to claim 2, wherein the endogenous immunoglobulin variable region genes include heavy chain variable region V_{H}, D_{H}, J_{H} and/or light chain variable region V_{L}, J_{L} of non-human mammalian cell immunoglobulin, wherein the light chain is kappa or lambda light chain.

4. The genetically engineered recombinant genome of non-human mammalian cell according to claim 3, wherein the coding and non-coding sequences for the human heavy chain functional V_{H}, D_{H}, J_{H} are from human chromosome 14 and the coding and non-coding sequences for the human light chain functional V_{L}, J_{L} are from human chromosome 2 or 22.

5. The genetically engineered recombinant genome of non-human mammalian cell according to claim 4, wherein the coding and non-coding sequences for the human heavy chain functional V_{H}, D_{H}, J_{H} comprise sequences between nucleotide positions 105863198 and 106879844 from human chromosome 14, all coordinates refer to the GRCh38.p13 version of the human genome database from ENSEMBL, preferably the coding and non-coding sequences for human heavy chain functional V_{H}, D_{H}, J_{H} comprise one or more of the V_{H} genes, preferably 10-41 V_{H} genes, more preferably 15-41 V_{H} genes, more preferably 18-41 V_{H} genes, more preferably 22-41 V_{H} genes, more preferably 25-41 V_{H} genes, such as 25, 26, 27, 28, 29, 30, 31, 32, 33, 34, 35, 36, 37, 38, 39, 40 or 41 V_{H} genes numbered as shown in the following table:

| | Name of human | | Start position | End position | Fragment |
|---|---|---|---|---|---|
| Sequence | heavy chain | Category of | of human | of human | size (base |
| number | functional V gene | V gene | chromosome 14 | chromosome 14 | pairs) |
| H1 | IGHV6-1 | Functional V gene | 105944896 | 105939715 | 5182 |
| H3 | IGHV1-2 | Functional V gene | 106001494 | 105986543 | 14952 |
| H5 | IGHV1-3 | Functional V gene | 106011882 | 106005056 | 6827 |
| H6 | IGHV4-4 | Functional V gene | 106025105 | 106011883 | 13223 |
| H7 | IGHV7-4-1 | Functional V gene | 106037862 | 106025106 | 12757 |
| H8 | IGHV2-5 | Functional V gene | 106039632 | 106037863 | 1770 |
| H10 | IGHV3-7, | Functional V gene | 106088082 | 106062110 | 25973 |
| | IGHV1-8, | | | | |
| | IGHV3-9 | | | | |
| H12 | IGHV3-11 | Functional V gene | 106120286 | 106116596 | 3691 |
| H14 | IGHV3-13 | Functional V gene | 106142246 | 106129501 | 12746 |
| H16 | IGHV3-15 | Functional V gene | 106163495 | 106153583 | 9913 |
| H18 | IGHV1-18 | Functional V gene | 106196658 | 106184860 | 11799 |
| H20 | IGHV3-20 | Functional V gene | 106212785 | 106210897 | 1889 |
| H22 | IGHV3-21 | Functional V gene | 106257722 | 106235023 | 22700 |
| H24 | IGHV3-23 | Functional V gene | 106276506 | 106268567 | 7940 |
| H25 | IGHV1-24 | Functional V gene | 106288923 | 106276507 | 12417 |
| H27 | IGHV2-26 | Functional V gene | 106308995 | 106301356 | 7640 |
| H29 | IGHV4-28 | Functional V gene | 106331105 | 106324215 | 6891 |
| H31 | IGHV3-30 | Functional V gene | 106344384 | 106335041 | 9344 |
| H33 | IGHV4-31 | Functional V gene | 106356144 | 106349244 | 6901 |
| H36 | IGHV3-33 | Functional V gene | 106369097 | 106360244 | 8854 |
| H38 | IGHV4-34 | Functional V gene | 106377231 | 106373622 | 3610 |
| H40 | IGHV4-39 | Functional V gene | 106425408 | 106421670 | 3739 |
| H42 | IGHV3-43 | Functional V gene | 106472846 | 106470224 | 2623 |
| H44 | IGHV1-45 | Functional V gene | 106511075 | 106506957 | 4119 |
| H45 | IGHV1-46 | Functional V gene | 106515891 | 106511076 | 4816 |
| H47 | IGHV3-48 | Functional V gene | 106556896 | 106537771 | 19126 |
| H48 | IGHV3-49 | Functional V gene | 106564377 | 106556897 | 7481 |
| H50 | IGHV5-51 | Functional V gene | 106583409 | 106578703 | 4707 |
| H52 | IGHV3-53 | Functional V gene | 106599595 | 106592637 | 6959 |
| H54 | IGHV1-58 | Functional V gene | 106627209 | 106622318 | 4892 |
| H55 | IGHV4-59 | Functional V gene | 106639079 | 106627210 | 11870 |
| H56 | IGHV4-61 | Functional V gene | 106643021 | 106639080 | 3942 |
| H58 | IGHV3-64 | Functional V gene | 106666004 | 106657684 | 8321 |
| H60 | IGHV3-66 | Functional V gene | 106680592 | 106674976 | 5617 |
| H62 | IGHV1-69 | Functional V gene | 106770537 | 106762053 | 8485 |
| H63 | IGHV2-70 | Functional V gene | 106775077 | 106770538 | 4540 |
| H65 | IGHV3-72 | Functional V gene | 106802652 | 106790653 | 12000 |
| H66 | IGHV3-73 | Functional V gene | 106810400 | 106802653 | 7748 |
| H67 | IGHV3-74 | Functional V gene | 106822782 | 106810401 | 12382 |

6. The genetically engineered recombinant genome of non-human mammalian cell according to claim 4, wherein the coding and non-coding sequences for human light chain functional V_{L}, J_{L} comprise sequences between nucleotide positions 88860568 and 90235398 from human chromosome 2, or sequences between nucleotide positions 22023114 and 22922913 from human chromosome 22, wherein all coordinates refer to the GRCh38.p13 version of the human genome database from ENSEMBL, preferably coding and non-coding sequences for the human light chain functional V_{L}, J_{L} comprise one or more of the V region genes numbered as shown in the following table:

| | Name of human | | Start position | End position | Fragment |
|---|---|---|---|---|---|
| Sequence | kappa light chain | Category of | of human | of human | size (base |
| number | proximal V Gene | V gene | chromosome 2 | chromosome 2 | pairs) |
| K1 | IGKV4-1 | Functional V gene | 88861968 | 88886183 | 24216 |
| K2 | IGKV5-2 | Functional V gene | 88886184 | 88897814 | 11631 |
| K4 | IGKV1-5 | Functional V gene | 88966231 | 88947271 | 18961 |
| K5 | IGKV1-6 | Functional V gene | 88978388 | 88966232 | 12157 |
| K7 | IGKV1-8 | Functional V gene | 89009981 | 88992379 | 17603 |
| K8 | IGKV1-9 | Functional V gene | 89019913 | 89009982 | 9932 |
| K10 | IGKV3-11 | Functional V gene | 89040193 | 89027141 | 13053 |
| K11 | IGKV1-12 | Functional V gene | 89045910 | 89040194 | 5717 |
| K13 | IGKV3-15 | Functional V gene | 89099828 | 89085147 | 14682 |
| K14 | IGKV1-16 | Functional V gene | 89117311 | 89099829 | 17483 |
| K15 | IGKV1-17 | Functional V gene | 89128657 | 89117312 | 11346 |
| K17 | IGKV3-20 | Functional V gene | 89159022 | 89142544 | 16479 |
| K18 | IGKV6-21 | Functional V gene | 89170744 | 89159023 | 11722 |
| K20 | IGKV2-24 | Functional V gene | 89192412 | 89176298 | 16115 |
| K22 | IGKV1-27 | Functional V gene | 89221667 | 89213393 | 8275 |
| K23 | IGKV2-28 | Functional V gene | 89234120 | 89221668 | 12453 |
| K25 | IGKV2-30 | Functional V gene | 89252117 | 89244751 | 7367 |
| K27 | IGKV1-33 | Functional V gene | 89275176 | 89267971 | 7206 |
| K29 | IGKV1-39 | Functional V gene | 89330085 | 89319595 | 10491 |
| K30 | IGKV2-40 | Functional V gene | 89333431 | 89330086 | 3346 |
| | Name of human | | Start position | End position | Fragment |
|---|---|---|---|---|---|
| Sequence | lambda light | Category of | of human | of human | size (base |
| number | chain V Gene | V gene | chromosome 22 | chromosome 22 | pairs) |
| L1 | IGLV3-1 | Functional V gene | 22873533 | 22881431 | 7898 |
| L3 | IGLV4-3 | Functional V gene | 22857100 | 22872074 | 14974 |
| L5 | IGLV2-8 | Functional V gene | 22819792 | 22823328 | 3536 |
| L6 | IGLV3-9 | Functional V gene | 22812321 | 22819791 | 7470 |
| L7 | IGLV3-10 | Functional V gene | 22793043 | 22812320 | 19277 |
| L8 | IGLV2-11 | Functional V gene | 22772622 | 22793042 | 20420 |
| L9 | IGLV3-12 | Functional V gene | 22762614 | 22772621 | 10007 |
| L11 | IGLV2-14 | Functional V gene | 22755877 | 22759250 | 3373 |
| L13 | IGLV3-16 | Functional V gene | 22739231 | 22747960 | 8729 |
| L15 | IGLV2-18 | Functional V gene | 22721195 | 22735128 | 13933 |
| L16 | IGLV3-19 | Functional V gene | 22715483 | 22721194 | 5711 |
| L18 | IGLV3-21 | Functional V gene | 22704858 | 22713238 | 8380 |
| L20 | IGLV2-23 | Functional V gene | 22695110 | 22698460 | 3350 |
| L22 | IGLV3-25 | Functional V gene | 22685389 | 22687320 | 1931 |
| L24 | IGLV3-27 | Functional V gene | 22664971 | 22668847 | 3876 |
| L26 | IGLV1-36 | Functional V gene | 22428075 | 22432504 | 4429 |
| L27 | IGLV5-37 | Functional V gene | 22426318 | 22428074 | 1756 |
| L29 | IGLV1-40 | Functional V gene | 22404761 | 22410321 | 5560 |
| L31 | IGLV7-43 | Functional V gene | 22381387 | 22395528 | 14141 |
| L32 | IGLV1-44 | Functional V gene | 22376545 | 22381386 | 4841 |
| L33 | IGLV5-45 | Functional V gene | 22370127 | 22376544 | 6417 |
| L34 | IGLV7-46 | Functional V gene | 22358302 | 22370126 | 11824 |
| L35 | IGLV1-47 | Functional V gene | 22353473 | 22358301 | 4828 |
| L37 | IGLV9-49 | Functional V gene | 22327856 | 22343773 | 15917 |
| L39 | IGLV1-51 | Functional V gene | 22319266 | 22323008 | 3742 |
| L40 | IGLV5-52 | Functional V gene | 22220173 | 22319265 | 99092 |
| L42 | IGLV10-54 | Functional V gene | 22202201 | 22215310 | 13109 |
| L44 | IGLV6-57 | Functional V gene | 22182891 | 22196315 | 13424 |
| L46 | IGLV4-60 | Functional V gene | 22099252 | 22162720 | 63468 |
| L47 | IGLV8-61 | Functional V gene | 22087064 | 22099251 | 12187 |
| L49 | IGLV4-69 | Functional V gene | 22026593 | 22031511 | 4918 |

7. The genetically engineered recombinant genome of non-human mammalian cell according to claim 1, wherein the endogenous immunoglobulin variable region genes are partially or entirely deleted, the human immunoglobulin heavy chain variable region genes are inserted at a location 3 KB upstream to 3 KB downstream from the deleted endogenous immunoglobulin heavy chain variable region, and the human immunoglobulin light chain variable region genes are inserted at a location 3 KB upstream to 3 KB downstream from the deleted endogenous immunoglobulin kappa light chain variable region,
preferably, the number of pseudogenes and/or open reading frame genes of the human immunoglobulin variable region genes knocked out (or, partial or entirely knocked out) should be sufficient such that the length of the human immunoglobulin heavy chain, Lambda light chain variable region genes inserted into the genome of the non-human mammalian cell is 10%-50%, preferably 12%-47%, preferably 14%-45%, preferably 15%-43%, more preferably 16%-40%, more preferably 16.10%, 18%, 18.50%, 20%, 25%, 30%, 31%, 31.75%, 35%, 38% or 38.06% of the total length of the human immunoglobulin heavy chain, Lambda light chain variable region genes before the knockout of the pseudogenes and/or open reading frame genes, respectively; and/or
the length of the human immunoglobulin kappa light chain variable region genes inserted into the genome of the non-human mammalian cell is 35%-65%, preferably 37%-63%, preferably 38%-61%, preferably 40%-60%, preferably 42%-58%, preferably 45%-57%, preferably 47%-56%, more preferably 50%-55%, such as 51%, 52%, 53%, 53.08% or 54% of the total length of the human immunoglobulin kappa light chain variable region gene before the knockout of the pseudogenes and/or open reading frame genes.

8. The genetically engineered recombinant genome of non-human mammalian cell according to claim 1, wherein the non-human mammalian cell is a mouse embryonic stem cell and the deleted endogenous immunoglobulin heavy chain variable region is located between positions 113428530 and 116027502 on mouse chromosome 12; the deleted endogenous immunoglobulin kappa light chain variable region is located between positions 67536984 to 70723924 on mouse chromosome 6; the deleted endogenous immunoglobulin lambda light chain variable region is located between positions 19065021 to 19260700 on mouse chromosome 16;
wherein the mouse genome chromosomal location coordinates refer to the locations of version GRCm38.p6 of C57BL/6J mouse genome database from ENSEMBL;
preferably, the insertion site of the human immunoglobulin heavy chain variable region genes is at position 113428513 on mouse genomic chromosome 12; the insertion site of the human immunoglobulin kappa light chain variable region genes is at position 70723924 on mouse genomic chromosome 6; the insertion site of the human immunoglobulin lambda light chain variable region genes is at position 70726758 on mouse genomic chromosome 6.

9. A non-human mammalian cell comprising the genetically engineered recombinant genome of non-human mammalian cell according to claim 1.

10. The non-human mammalian cell according to claim 9, wherein the cell is a non-human mammalian embryonic stem cell, preferably, the non-human mammalian embryonic stem cell is a mouse embryonic stem cell, a rat embryonic stem cell, or a rabbit embryonic stem cell.

11. A method of producing the non-human mammalian cell of claim 9, comprising:
a) introducing identical orientated and compatible recombinase targeting sites to upstream and downstream respectively of the immunoglobulin variable region gene in the genome of a non-human mammalian cell;
b) introducing a specific recombinase capable of recognizing the recombinase sites of step a), allowing recombination event to occur between the two recombinase targeting sites of step a) resulting in partial or entire deletion of the endogenous immunoglobulin variable region genes of the non-human mammalian cell;
c) providing a targeting vector comprising part or all of the human immunoglobulin variable region, wherein the targeting vector contains human functional variable region genes and part or all of the pseudogenes and/or open reading frames are knocked out; the human functional variable region genes comprise coding and non-coding sequences for human heavy chain functional V_{H}, D_{H}, J_{H}, or coding and non-coding sequences for human light chain functional V_{L}, J_{L}; and the light chain is a kappa or lambda light chain;
d) introducing the targeting vector of step c), resulting in the replacement of the deleted non-human mammalian cell endogenous immunoglobulin gene of step b) by the human immunoglobulin variable region gene in step c) in the non-human mammalian cell;
e) generating the non-human mammalian cell comprising human immunoglobulin variable region genes in the genome from step d).

12. The method according to claim 11, wherein the targeting vector is selected from BAC vector or YAC vector.

13. The method according to claim 11, wherein the targeting vector in step c) is constructed in E. coli or yeast cells.

14. A targeting vector, comprising human immunoglobulin variable region genes, wherein a part or all of the pseudogenes and/or open reading frames of the human immunoglobulin variable region genes are knocked out, the human immunoglobulin variable region genes comprise coding and non-coding sequences for human heavy chain functional V_{H}, D_{H}, J_{H}, or coding and non-coding sequences for human light chain functional V_{L}, J_{L}, the light chain is a kappa or lambda light chain.

15. The targeting vector according to claim 14, wherein the targeting vector is selected from BAC vector or YAC vector.

16. A method of generating a non-human mammal expressing an antibody with fully human variable region(s), comprising introducing the non-human mammalian cell according to claim 9 into the utero of a female wild-type non-human mammal, selecting the progeny chimeric non-human mammal as F0 generation non-human mammal.

17. The method of generating a non-human mammal expressing an antibody with fully human variable region(s) according to claim 16, wherein the non-human mammalian cells are screened before introducing the non-human mammal cells into the utero of a female wild-type non-human mammal, to obtain a non-human mammalian cell clone having no increase or a decrease in chromosome number, the non-human mammalian cell clone are transplanted into a wild-type non-human mammalian embryonic blastocyst cavity, and the blastocyst are transplanted into a pseudopregnant female wild-type non-human mammalian utero.

18. The method of generating a non-human mammal expressing an antibody with fully human variable region(s) according to claim 16, wherein the F0 generation non-human mammal is propagated with a wild-type non-human mammal to obtain a stably inheritable F1 generation non-human mammal having human immunoglobulin variable region genes inserted at specified positions.

19. A method of generating a non-human mammal expressing an antibody with fully human variable region(s) according to claim 16, wherein the non-human mammal is a mouse, a rat or a rabbit and the non-human mammalian cell is a mouse embryonic stem cell, a rat embryonic stem cell or a rabbit embryonic stem cell.

20. A non-human mammal prepared by the method of generating a non-human mammal expressing an antibody with fully human variable region(s) according to claim 16, preferably, the non-human mammal is a mouse, rat or rabbit.

21-22. (canceled)

23. An antibody or an antibody fragment with fully human variable region produced by the non-human mammal of claim 20, or a derivative drug or pharmaceutical composition comprising the antibody or antibody fragment.
